SEQUENCE LISTING (5 sequences)

SEQ ID NO: 1 - protein, 27 aa, Artificial Sequence
  Synthetic peptide containing epitope sequences of SEQ ID NO2 and SEQ ID NO4.
DAEFRHDKMQQNGYENPTYKFFEQMQN

SEQ ID NO: 2 - protein, 20 aa, Artificial Sequence
  Synthetic peptide containing residues 676-695 of APP695.
KMQQNGYENPTYKFFEQMQN

SEQ ID NO: 3 - protein, 32 aa, Artificial Sequence
  Synthetic peptide containing residues 644-676 of APP695.
LVMLKKKQYTSIHHGVVEVDAAVTPEERHLSK

SEQ ID NO: 4 - protein, 7 aa, Artificial Sequence
  Synthetic peptide containing epitope for JRF/ABN/25 antibody.
DAEFRHD

SEQ ID NO: 5 - protein, 40 aa, Artificial Sequence
  Synthetic peptide recognized by JRF/ABtot/17 antibody.
DAEFRHDSGYEVHHQKLVFFAEDVGSNKGAIIGLMVGGVV

(19) United States
(12) Patent Application Publication
(10) Pub. No.: US 2004/0253647 A1
(43) Pub. Date: Dec. 16, 2004

(54) Cell-based high-throughput screening methods

(76) Inventors: Paul M. Mathews, Irvington-on-Hudson, NY (US); Ralph A. Nixon, Tarrytown, NY (US); Stephen D. Schmidt, Nutley, NJ (US); Ying Jiang, Valhalla, NY (US)

Correspondence Address: MORGAN & FINNEGAN, L.L.P., 3 WORLD FINANCIAL CENTER, NEW YORK, NY, 10281-2101, US

(21) Appl. No.: 10/481,954

(22) PCT Filed: Jun. 26, 2002
(86) PCT No.: PCT/US02/20267

Related U.S. Application Data
(60) Provisional application No. 60/300,959, filed on Jun. 26, 2001.

(51) Int. Cl. G01N 33/53; G01N 33/567

(52) U.S. Cl. 435/7.2

(57) ABSTRACT

The present invention describes cell-based screening methods that allow for the elimination of false-positive results due to nonspecific toxicity of test compounds, while detecting those compounds that specifically modulate cellular metabolites in metabolic pathways associated with diseases and disorders, with particular regard to Alzheimer's disease. The methods are particularly suited to high-throughput screening techniques to identify compounds (drugs) that are effective in a cell-based system. The methods of the invention involve determining within a cell system the levels of both a metabolic precursor and a metabolite of interest and have broad application in high-throughput drug discovery and identification, particularly for precursor and metabolite molecules which are associated with disease and disease progression, such as Alzheimer's disease.

The work described herein is supported in part by grant no. P01 AG17617 from The National Institutes of Health, National Institute of Aging.

### FIELD OF THE INVENTION

The present invention relates to cell-based screening methods, particularly suitable for high-throughput screening systems, for use in the identification and discovery of compounds (drugs) that can serve as therapeutics in the treatment and/or prevention of diseases and disorders.

### BACKGROUND OF THE INVENTION

An invariant feature of Alzheimer's disease is the deposition of the small; i.e., approximately 40 to 42 residues, Aβ peptide as insoluble β-amyloid plaque in the brain parenchyma. Aβ is generated by proteolysis of the approximately 100 kDa amyloid precursor protein (APP), a broadly expressed type-1 transmembrane. protein that is found primarily in the trans-Golgi network (TGN) and at the cell surface (reviewed in B. De Strooper and W. Annaert, 2000, “Proteolytic processing and cell biological functions of the amyloid precursor protein.” J. Cell. Sci., 113(Pt 11)(7):1857-1870). The β-amyloid precursor protein APP is further, described in D. J. Selkoe et al., 1988, Proc. Natl. Acad. Sci. USA., 85(19):7341-7345; R. E. Tanzi et al., 1988, Nature, 331(6156):528-530; and E. Levy et al., 1990, Science, 248(4959):1124-1126.

The β-cleavage of APP occurs within the lumenal/extracellular domain of APP and generates two APP fragments: (i) a large, soluble amino-terminal fragment (sAPP) that is secreted from the cell, and (ii) a transmembrane, carboxy-terminal fragment (βCTF)that remains associated with the cell. This βCTF contains 99 amino acids, comprises the whole Aβ peptide, and has a molecular weight of approximately 10 kDa.

An alternative pathway involves the cleavage of APP sixteen residues downstream from the β-cleavage site at the α-cleavage site. Like β-cleavage, α-cleavage generates a secreted APP (sAPP) fragment that is secreted from the cell and an αCTF (of 84 residues and approximately 8 kDa) that remains membrane associated. α-cleavage occurs within the Aβ peptide sequence, and as such, prevents the generation of Aβ from a given APP molecule. Aβ is generated from the βCTF by an intra-membrane cleavage (γ-cleavage) that occurs primarily at 40 residues, and to a lesser extent, at 42 residues downstream from the β-cleavage site, releasing Aβ1-40 or Aβ1-42.

Recently, much progress has been made in identifying the major proteases/protease complexes responsible for β- and γ-cleavage (i.e., the β- and γ-secretases). The BACE proteases, which are members of a family of transmembrane aspartyl proteases, were first identified by Citron and colleagues (R. Vassar et al., 1999, Science, 286(5440):735-741) and appear to account for much of the β-secretase activity within a cell. BACE has an endosomal-lysosomal pattern of distribution, as well as an acidic pH optimum (R. Vassar et al., 1999, Science, 286(5440):735-741; A. Capell et al., 2000, J. Biol. Chem., 275(40):30849-30854; and J. Walter et al., 2001, J. Biol. Chem., 276(18):14634-14641). In addition, BACE-mediated cleavage of APP in the endocytic system is consistent with prior work that has identified, through various trafficking mutants of APP, the early endosome as an important site for Aβ generation (E. H. Koo and S. L. Squazzo 1994, J. Biol. Chem., 269(26):17386-17389; R. Perez et al., 1999, J. Biol. Chem., 274(27):18851-18856; S. Soriano et al., 1999, J. Biol. Chem., 274(45):32295-32300; and A. M. Cataldo et al., 2000, Am. J. Pathol., 157(1):277-286).

The presenilin (PS) proteins play an intimate role in γ-cleavage: expression of familial AD-causing mutant presenilin increases the production of Aβ terminating at residue 42 (D. R. Borchelt et al., 1996, Neuron, 17(5):1005-1013; K. Duff et al., 1996, Nature, 383(6602):710-713). The PS-null phenotype includes the inability of the cell to generate Aβ and the intracellular accumulation of CTFs (J. Shen et al., 1997, Cell, 89(4):629-639). Recent work has directly implicated PS itself as the γ-secretase (M. S. Wolfe et al., 1999, Nature, 398(6727):513-517), although other proteins within the PS complex, such as nicastrin (G. Yu et al., 2000, Nature, 407(6800):48-54), may well be directly involved in γ-cleavage.

While BACE and the PS complex may be the major β- and γ-secretases, substantial experimental work has implicated other proteases, particularly those of the lysosome (R. A. Nixon et al., 2000, Neurochem. Res., 25(9-10):1161-1172; and P. M. Mathews et al., 2002, J. Biol. Chem., 277:5299-5307). The relative contribution of these other proteases may be increased in AD due to their mis-trafficking to endocytic compartments (A. M. Cataldo et al., 1995, Neuron, 14(3):671-680; A. M. Cataldo et al., 1996, Adv. Exp. Med. Biol., 389:271-280; A. M. Cataldo et al., 1997, Neuroscience, 17(16):6142-6151.; A. M. Cataldo et al., 2000, Am. J. Pathol., 157(1): 277-286; R. A. Nixon et al., 2000, Neurochem. Res., 25(9-10):1161-1172; O. M. Grbovic et al., 2001, Society for Neuroscience annual meeting 2001; and patent application U.S. Ser. No. 09/560,124 “Methods for the identification of compounds for the treatment of Alzheimer's disease”; to R. A. Nixon et al., filed Apr. 28, 2000).

Substantial effort is currently directed toward the identification of drugs that inhibit the various proteolytic events giving rise to Aβ. Existing approaches are typically based upon screens that employ the purified enzyme. The limitations of these approaches are numerous, and include (1) the difficulty of translating purely in vitro inhibitors into in vivo use; (2) inherent assumptions made about the identity of the protease target; and perhaps most significantly, (3) such approaches do not detect compounds that will affect Aβ generation in the complex environment of the cell, where protein trafficking, local environment (e.g. pH), and complex enzyme interactions, among other factors, are relevant. An example of this is the mechanism by which estrogen replacement therapy may be protective against AD: estrogen is thought to act by promoting the intracellular trafficking of APP along non-β generating pathways (A. B. Jaffe et al., 1994, J. Biol. Chem., 269(18):13065-13068; H. Xu et al., 1998, Nat. Med., 4(4):447-451; and S. Gandy and S. Petanceska, 2000, Biochim. Biophys. Acta, 1502(1):44-52).

The screening method provided by the present invention overcomes many of the labor-intensive and technical limitations of cell-based screening, while at the same time allows the user to take advantage of the complexity of cellular responses that may be of benefit in treating diseases and disorders that presently are difficult to treat, for example, AD, Parkinson's disease, Huntington's disease, lysosomal storage disorders, prion diseases, the tau-based neurodegenerative disorders (the tauopathies), and other non-AD amyloidoses.

### SUMMARY OF THE INVENTION

The present invention provides new cell-based screening methods and techniques that are particularly suited for high-throughput screening analyses for the identification and discovery of new drugs for treating diseases and disorders, preferably diseases and disorders associated with metabolic and/or proteolytic pathways in which one or more metabolites is generated from a metabolic precursor or precursors, and in which an increase or decrease of the one or more metabolites in the pathway is associated with disease.

The cell-based screening methods according to the present invention provide the advantage of dramatically reducing the number of false-positive results that are typically obtained in cell-based high-throughput assay schemes. Use of the present invention advantageously allows the identification of compounds that specifically modulate a metabolic and/or proteolytic pathway. Moreover, the present inventive methods provide the ability to identify those compounds that are generally and non-specifically toxic to cells undergoing high-throughput screening analysis, which, in other assays, could be erroneously identified as potential therapeutics. Thus, the present invention allows for the elimination of compounds as potential therapeutics if such compounds are non-specifically and/or generally toxic to cells.

It is one aspect of the present invention to provide a versatile cell-based screening method in which the levels of both a metabolic precursor protein (e.g., APP) and a corresponding metabolite product (e.g., βCTF) are determined, preferably a biologically meaningful metabolite product and preferably in a high-throughput screening system, so as to reduce the number of false-positives that are detected and yield an efficient and reliable screening technique. Also in accordance with the present invention, detection of the levels of different conformation states of a precursor protein in a pathway associated with a disease or disorder is provided by the screening methods described herein. In such methods, the metabolic precursor and metabolite can be, respectively, the different conformation states of the same protein, for example, as in the prion disease. As another example, the precursor protein can be unphosphorylated and the metabolite is a phosphorylated form of the precursor protein.

Such screening techniques allow for the identification of compounds that ultimately modulate, e.g., reduce or inhibit or increase or augment, a cellular processing event (e.g., β-secretase cleavage) upon a metabolic precursor protein (e.g., APP), thereby influencing the generation of one or more metabolites involved in progression of disease, such as Alzheimer's disease.

It is a particular aspect of the present invention to provide a specific and sensitive assay/detection system for β-cleavage inhibitors to discover or identify agents and new drugs for the treatment, therapy, and/or prevention of Alzheimer's disease, preferably in conjunction with high-throughput screening techniques. The screening methods allow for the detection of inhibitors of a critical proteolytic event in the generation of Aβ, which in accordance with this invention can be used in drug development for treatments of Alzheimer's disease and/or for treatments of diseases and conditions related to Alzheimer's disease, e.g., β-amyloid related diseases.

It is yet another aspect of the present invention to provide highly sensitive and specific novel immunoassays, namely ELISAs, to detect cell-associated proteolytic or cleavage metabolites of the amyloid precursor protein APP, preferably in conjunction with high-throughput screening techniques. In accordance with this invention, one novel ELISA allows for the specific detection of a key peptide fragment, βCTF, which is generated along the pathway to the small peptide Aβ, resulting from the proteolytic processing of APP, and which is believed to be central to the pathogenesis of Alzheimer's disease. A second novel ELISA according to this invention allows for the detection of APP holoprotein and all known APP CTFs (i.e., the αCTF, the βCTF and the γCTF). These ELISAs can be used in combination as powerful tools to determine the metabolism of APP along Aβ-generating pathway in a living cell treated with a compound that may inhibit β-secretase cleavage of APP.

Further aspects, features and advantages of the present invention will be better appreciated upon a reading of the detailed description of the invention when considered in connection with the accompanying figures/drawings.

### BRIEF DESCRIPTION OF THE FIGURES

FIG. 1: Monoclonal antibody specificity for APP holoprotein and CTFs. An L cell line overexpressing human APP (L/APP) was metabolically labeled for 15 minutes and chased for 1 hour as indicated. Cells were pretreated with the indicated calpain inhibitors for 3 hours prior to metabolic labeling, as well as during labeling and chase. Cell lysates were prepared, and equal volumes were immunoprecipitated with one of three monoclonal antibodies: C1/6.1, which recognizes an epitope within the 20 carboxy-terminal-most residues of APP; JRF/AβN/25, which recognizes an epitope within residues 1-7 of Aβ; and JRF/Aβtot/17, which recognizes an epitope within residues 1-16 of Aβ. (Table 1, Example 1). Labeled, immunoprecipitated proteins were sized on SDS-PAGE and detected as described in Example 1, Methods. Arrows indicate the APP holoprotein (APP_{fl}) and the α- and β-cleaved CTFs (αCTF and βCTF, respectively).

FIGS. 2A and 2B: Detection of APP holoprotein and αCTFs, βCTFs, and γCTFs with C1/6.1. The L cell line overexpressing APP was metabolically labeled and chased for the indicated times. Calpeptin treatment was performed as described for FIG. 1. Cell lysates were immunoprecipitated with C1/6.1 monoclonal antibody as described in Example 2. FIG. 2A depicts a short exposure showing the turnover of the APP holoprotein (APP_{fl}). FIG. 2B depicts a longer exposure showing the APP holoprotein and the α-, β-, and γ-cleaved CTFs (αCTF, βCTF, γCTF; indicated by asterisks and arrows).

FIG. 3: βCTF ELISA. ELISA plates were coated with C1/6.1 monoclonal antibody, a synthetic peptide standard, DAEFRHDKMQQNGYENPTYKFFEQMQN, (SEQ ID NO:1), was bound, and bound peptide was detected with JRF/AβN/25 as described in Example 1, Methods, and in Example 3. The optical density (at 450 nm) was graphed as a function of the femtomoles/ml of peptide added to each well. Values are the mean of two measurements.

FIG. 4: Quantitative detection of βCTFs isolated from cells. Human APP overexpressing L cells (L/APP) were grown and human APP₆₉₅ expression was induced as described in Example 1, Methods. Detergent lysates were prepared from a control well and a well treated with 10 82 M calpeptin for 6 hours prior to extraction. βCTF levels were determined by ELISA using C1/6.1 as the capture antibody and JRF/AβN/25 as the detecting antibody. Values are reported as the mean of duplicate, ELISA readings ±SD. (Example 3).

FIGS. 5A and 5B: Quantitative ELISA detection of changes in βCTF levels following pharmacological and genetic manipulations. (Example 3). FIG. 5A presents a Western blot analysis using C1/6.1 of L/APP cells grown as described. Lane 1 was loaded with untreated L/APP cells; lane 2, cells treated with 10 μm; calpeptin for 6 hours prior to extraction; lane 3, cells transiently transfected with 0.5 μg/well rab5 cDNA 48 hours prior to extraction; lane 4, cells transiently transfected with 1.0 μg/well rab5 cDNA using fugene; lane 5, cells transiently transfected with 1.0 ug/well rab5 cDNA using lipofectAMINE (Gibco/BRL, Gaithersburg, Md.). The APP holoprotein (APP_{fl}) and CTFs are indicated. FIG. 5B presents the levels of βCTFs detected from these lysates by ELISA. Unlike calpeptin treatment, rab5 overexpression increased βCTF levels in the cells, while not increasing αCTF levels (O. M. Grbovic et al., 2001, Society for Neuroscience annual meeting 2001).

FIG. 6: APP/total CTF ELISA. ELISA plates were coated with C1/6.1, the GST-βPP672-770 fusion protein standard was bound, and bound GST-βPP672-770 was detected with C2/7.1 as described in Example 1, Methods, and in Example 4. The optical density (at 450 nm) was graphed as a function of the femtomoles/ml of fusion protein added to each well. Values are the mean of two measurements.

FIGS. 7A and 7B: Quantification by ELISA of APP holoprotein and all CTFs in cells. Equal numbers of parental L cells and L/APP cells were plated and APP₆₉₅ expression was induced. Cell lysates were prepared and, in FIG. 7A, analyzed by Western blot using C1/6.1 monoclonal antibody (APP holoprotein is indicated). In FIG. 7B, similar cell lysates were analyzed by ELISA using C1/6.1 as the capture antibody and C2/7.1 for detection. As indicated, cells were treated with 10 μM calpeptin for 6 hours prior to extraction. (Example 4).

FIGS. 8A and 8B: Cyclohexamide treatment reduces both βCTF and APP/total CTF levels. L/APP cells were plated at equal density and APP expression was induced. During the final 6 hours of induction, cells were treated with 10 μM calpeptin or 75 μg/ml cyclohexamide as indicated. Lysates were prepared and analyzed by ELISA for βCTF levels (FIG. 8A) and for APP/total CTF levels (FIG. 8B) as described in Example 1, Methods, and in Example 5. Results are the mean of duplicate measurements and are expressed as fmole/ml of cell lysate relative to standard.

FIGS. 9A-9D: Schematic of high-throughput screening protocol. (FIG. 9A): ELISA wells are-initially coated with C1/6.1 monoclonal antibody, which specifically binds to the intracellular, carboxy-terminus of APP (the capture antibody), or antibodies which specifically bind to the APP cellular metabolites (carboxy-terminal fragments of APP, i.e., αCTF, βCTF, or γCTF). (FIG. 9B): Cells of interest (i.e., mammalian, including human, cells and cell lines, cell types such as neuroblastomas, cells transfected to express APP or other proteins linked to AD pathogenesis; or cells genetically modified to mimic aspects of AD pathobiology) are seeded into the well, allowed to settle, and the test compound added.

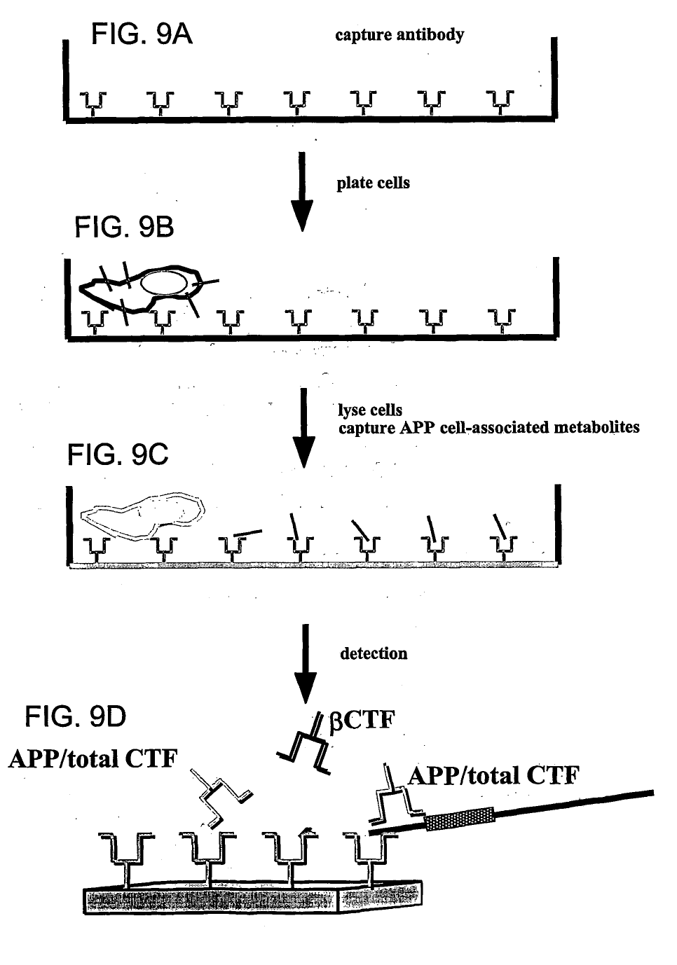

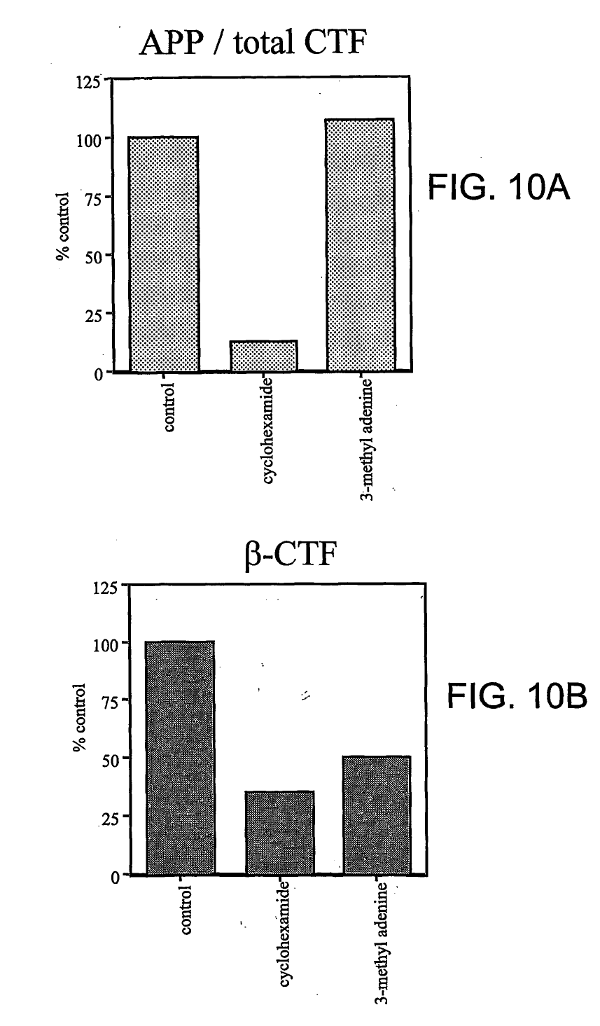

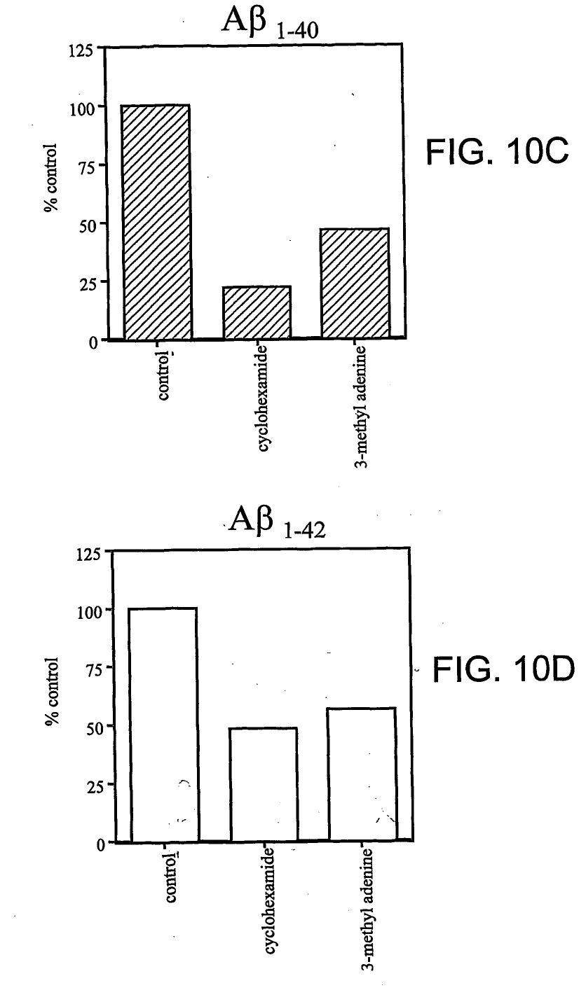

Due to the rapid turnover of the βCTF compared with the turnover of Aβ secreted into the growth medium, drug treatment can be for a much shorter time period (e.g., about 1-2 hours) than that required if Aβ were to be measured. Moreover, no changes of the growth media are required. (FIG. 9C): Cells are disrupted (lysed) in situ and the detergent extracted-APP fragments allowed to bind to the pre-coated capture antibody(ies). Alternatively, cells are grown in separate wells and cell lysates are added to the capture antibody. Following washes, the two detection antibodies, which are differently labeled (e.g., with different fluorophores), are allowed to bind. (FIG. 9D): One of the detection antibodies, JRF/AβN/25 (black antibody), recognizes only βCTF and the other detection antibody C2/7.1 recognizes APP holoprotein and all APP CTFs, including the βCTF (gray antibody). The binding of these antibodies is then detected quantitatively using standard assays, such as a color reaction as described, for example, by C. Janus et al., 2000, Nature, 408(6815): 979-82, or by employing fluorophore-coupled antibodies and fluorescence detection.

The levels of βCTF and APP and its cleavage metabolites are determined, based on the intensity of a color reaction or fluorescence signals relative to control. A ratio of specific antibody binding to βCTF to specific antibody binding to APP and all APP cleavage metabolites e., αCTF, βCTF and γCTF) can be determined (ratio of black antibody to gray antibody) and compared to the same ratio in the control. If necessary or desired for technical reasons, detection using these two, antibodies can be performed in different wells, without compromising the ability of this screen to differentiate between compounds that specifically reduce β-cleavage and compounds that are simply toxic to the cells.

FIGS. 10A-10D: 3-methyl adenine selectively inhibits βCTF generation and Aβ production without reducing total cellular APP. L/APP cells were plated at equal density and APP expression was induced. Cells were then incubated for 6 hours without treatment, or with the addition of 10 μg/ml cyclohexamide or 10 μg/ml 3-methyl adenine (3MA). APP/total CTF levels (FIG. 10A) and βCTF levels (FIG. 10B) in cell lysates were then determined by ELISA. Aβ1-40 (FIG. 10C) and Aβ1-42 (FIG. 10D) levels secreted into the growth media during this 6 hour incubation were determined by ELISA (C. Janus et al., 2000, Nature, 408(6815):979 982). Data are expressed as a percentage of, the level seen in untreated control cells for each assay.

### DESCRIPTION OF THE INVENTION

The present invention describes a sensitive and specific screening method/system, which is also both efficient and economical, to determine the levels of both a metabolic precursor and its biologically relevant metabolite or product, preferably in cells undergoing testing for compounds or agents that modulate or affect the generation of the resulting metabolite or product from its metabolic precursor. By modulate is meant that the bioactivity of a molecule is altered, i.e., either decreased (i.e., reduced, inhibited, or blocked), or increased (i.e., activated, enhanced, or augmented). As used herein, compound refers to a biological or bioactive agent, or drug, or substance, or ingredient, or biomolecule, for example, as further described herein. Illustratively, the function or activity of a target molecule, or a metabolic or proteolytic process associated with a disease or disorder is modulated, for example, by being reduced, decreased, or inhibited; or increased, augmented, or enhanced.

In a preferred embodiment according to the present invention, the function or activity of a target molecule or metabolic or proteolytic process is reduced, decreased, or inhibited. Preferably, the screening method/system of this invention is performed using high throughput screening procedures, and more preferably is cell-based, thus providing applicability to many drug discovery schemes for various diseases and disorders having a detectable and assayable metabolic precursor (e.g., a protein) and its metabolite product (e.g., a proteolytic fragment of the protein) associated therewith.

More specifically, with particular regard to treatments and therapies for Alzheimer's disease, the present invention provides methods and procedures to identify, in an efficient and cost-effective manner, therapeutic agents, compounds, or drugs that ultimately reduce the amount of the Aβ product generated by a cell. As mentioned above, the Aβ peptide forms insoluble β-amyloid plaques in the brain parenchyma as part of the debilitating effects of Alzheimer's disease. The present methods allow the determination and employment of new treatments for, and/or the prevention of, Alzheimer's disease. The methods of the present invention also allow the screening and determination of molecules that modify proteolytic or metabolic pathways, or other cellular events, that affect, e.g., by reducing, the production of a metabolite. Thus, the cell-based methods of the invention can be advantageously employed to identify compounds or bioactive agents that modulate cellular processes that prevent an interaction between a protein, e.g., a proteolytic enzyme, and its target, e.g., a substrate, for example, β-secretase and APP.

High-throughput drug screening on living cells often generates an overwhelming number of false-positive hits, particularly when a reduction in an activity is being assayed. This is because many compounds and agents undergoing testing are simply toxic, and nonspecific toxicity frequently reduces the target activity. For this reason, high-throughput screening of living cells is rarely carried out when the desired outcome is a reduction in a particular cellular activity. As more particularly described herein, this is a challenging hurdle for Alzheimer's disease drug discovery, as compounds that reduce Aβ generation by a cell (D. J. Selkoe, 1999, Nature, 399(6738 Suppl): A23-31; D. J. Selkoe, 2001, Physiol. Rev., 81(2): 741-66) and/or reduce Aβ accumulation in the brain are likely to have treatment value (C. Janus et al., 2000, Nature, 408(6815): 979-82; D. Morgan et al., 2000, Nature, 408(6815): 982-985).

According to the present invention a novel screening method, preferably a cell-based method, has been developed that allows for the elimination of false-positive hits due to nonspecific toxicity while detecting particularly informative cellular metabolites that are generated in the pathway of products that are associated with or linked to various disease states, for example, along the pathway to Aβ generation associated with Alzheimer's disease. Overcoming the false-positives associated with cell toxicity makes high-throughput cell-based screening a practical, cost effective and appealing approach to identifying compounds that target molecules which are operative in the metabolic pathways that are associated with, and perhaps cause and/or exacerbate, a disease state.

Indeed, once a compound is identified as being effective in a cell-based system, it has a much greater probability of in vivo efficacy and translation to clinical practice than does a compound or other molecule identified, for example, using a purified enzyme, or a similar, less complex in vitro system.

A preferred embodiment of, the present invention is an enzyme linked immunosorbent assay (ELISA)-based method and approach that is specifically designed to screen for modulators or effectors, preferably, antagonists or inhibitors, of target proteolytic enzymes, such as the amyloid precursor protein β-secretases, e.g., the transmembrane aspartic protease BACE, (R. Vassar et al., 1999, Science, 286(5440): 735-741). The method according to the invention involves the detection of the amyloid precursor protein APP and its cleavage fragments (metabolites), namely, αCTF, βCTF and γCTF, alone or in combination.

The methods according to this invention are generally applicable to determining within a cell system both the levels of a metabolic precursor and the levels of a metabolite of interest. These methods allow the identification of modulators, e.g., inhibitors, blockers, or antagonists; or agonists or activators, that affect (e.g., inhibit the generation of a metabolite from a metabolic precursor, or e.g., increase the production or secretion of a precursor or protein) precursor and breakdown molecules involved in metabolic processes or pathways, including proteolytic pathways.

In addition to providing a screen for Alzheimer's disease therapeutics, as particularly exemplified herein, the present invention provides a way to screen in a cellular milieu for metabolites and their precursors that are associated with a number of other diseases and disorders. Nonlimiting examples of such diseases and disorders include lysosomal storage disorders, Parkinson's disease, Huntington's disease, neuronal ceroid lipfuscinoses, the tau-based neurodegenerative disorders (the tauopathies), and non-AD amyloidoses (e.g, inclusion body myositis) in which the enzymatic system that generates the amyloid or abnormally accumulated product is targeted, as well as other conditions in which there is the generation of an assayable metabolic breakdown protein or peptide product, or metabolite, derived from a precursor polypeptide. Additional examples include diseases in which a unique conformational state of protein accumulates, such as the prion diseases.

In such diseases and for the purposes of this invention, the normal, non-pathological conformation is the precursor, while the pathological conformational state, which can include toxic oligomeric peptides, is the metabolite, where both can be assayed using appropriate and specific probes, such as specific and detectable antibodies. A precursor to metabolite relationship can include other post-translational modifications, such as phosphorylation, where the precursor protein substrate and post-translationally modified polypeptide can both be assayed, e.g., as illustrated by tauopathies. In addition, metabolites in accordance with the present invention also include protein or peptide complexes (e.g., dimers, trimers, multimers), oligomers, polymers, oligomeric assemblages, or protein or peptide assemblies, for example, in beta-sheets or other arrangements, including, for example, protofibrillar and fibrillar molecules or macromolecules, e.g., Aβ, or a molecule resulting from the association of two or more peptides or proteins, such as a macromolecular complex. Preferably, the metabolite, or one or more portions thereof, is antigenic and detectable by an antibody.

In a preferred embodiment, highly sensitive and specific novel ELISAs, e.g., sandwich ELISAs, to detect cell-associated proteolytic or cleavage metabolites of the APP have been developed and are described herein, with particular regard to high-throughput screening methodologies. Much current research is directed at modifying the proteolytic processing of APP, which yields the small peptide Aβ, thought to be central to the pathogenesis of AD. One of the ELISAs described herein allows for the specific detection of a key peptide fragment generated along the pathway to Aβ- the βCTF. A second ELISA allows for the detection of APP holoprotein, and, optionally, all known APP CTFs, namely, the αCTF, βCTF and the γCTF. In combination, these ELISAs are powerful tools to determine the metabolism of APP along an Aβ-generating pathway in a living cell treated with compound that may inhibit β-secretase cleavage of APP.

Moreover, by combining the sandwich ELISAs into a single system, preferably a high-throughput system, compounds that are toxic to a cell can be rapidly distinguished from those that specifically reduce the critical APP proteolytic step. Toxic agents and compounds are likely to reduce the levels of APP holoprotein, as well as the CTF cleavage products that are detected in a cell-based screen, by such mechanisms as reducing cell growth, reducing cell viability, acting as protein biosynthesis poisons, or in other ways that globally compromise the cell's metabolism. Such a toxic effect would be detected by a reduced signal in an APP/total CTF ELISA.

However, a compound that specifically reduces βCTF generation and shows a reduction only in the βCTF ELISA signal is unlikely to reduce the signal from an APP/total CTF ELISA. Therefore, a high-throughput screen can readily be based upon a reduction in the βCTF ELISA signal resulting from treatment with a compound, relative to the signal from untreated cells, while treatment with the same compound shows no change in the APP/total CTF ELISA signal relative to untreated cells. Accordingly, compounds that specifically reduce βCTF generation will show a reduction of βCTF levels in the βCTF ELISA, and will not show a significant reduction in the levels of APP/total CTF ELISA. In contrast, generally toxic compounds are likely to lower both the βCTF ELISA level and the APP/total CTF ELISA level, as detected by reduced signal in an ELISA-based assay. Thus, the methods according to the present invention allow the skilled practitioner to differentiate β-cleavage-specific inhibitor compounds from compounds that are non-specifically cytotoxic, or toxic to cells in general.

An additional benefit for high-throughput screening is to overlay the above-described two ELISAs into a sequential assay that obviates the need for the use of parallel 96-well microtiter plates (see, e.g., FIGS. 9A-9D and description thereof. Such a protocol allows the method to be carried out in the same well in which the assay cells are grown using specific antibodies directed toward one or more target precursor proteins and one or more target metabolites. That the particular ELISAs described herein perform well using a single well in which the cells are grown is due in part to the unique intracellular APP epitopes detected by the C1/6.1 and C2/7.1 monoclonal antibodies. The production and screening of monoclonal antibodies that are specific for particular epitopes comprising a precursor protein such as APP and metabolite products are well known to those having skill in the art. Further, reducing the numbers of plates needed and assays performed has clear cost benefits and enhances the reliability of the assay, as each determination has a control measurement that is carried out within the same well.

In performing the cell-based methods of the present invention, cells that are undergoing testing and treatment with test compounds to identify modulators, preferably antagonist or inhibitor compounds, can include, without limitation, cultured or established mammalian cells or cell lines, including human cells or cell lines, nerve cells, neurons, neuroblastoma cells, cells transfected with a gene encoding amyloid precursor protein (APP) and which express APP, cells transfected with a gene encoding a protein linked to Alzheimer's disease pathogenesis (e.g., presenilin (R. Sherrington et al., 1995, Nature, 375(6534):754-760), and cells genetically modified to mimic aspects of Alzheimer's disease pathobiology, such as rab5 overexpression (O. M. Grbovic et al., 2001, Society for Neuroscience, annual meeting 2001).

Further examples of cells genetically modified to mimic aspects of Alzheimer's disease pathobiology include, but are not limited to, those described in patent application U.S. Ser. No. 09/560,124 “Methods for the identification of compounds for the treatment of Alzheimer's disease”; to R. A. Nixon et al., filed Apr. 28, 2000; in P. M. Mathews et al., 2001, “Accelerated Aβ generation in a cell model of Alzheimer's disease-related endosomal-lysosomal system upregulation.” Alzheimer's Disease: Advances in Etiology, Pathogenesis and Therapeutics (Iqbal K, Sisodia S S, Winblad B, editors), John Wiley & Sons, Ltd., Chichester, UK, 461-467; and P. M. Mathews et al., 2002, J. Biol. Chem., 277:5299-5307). Suitable cells may be vertebrate, preferably mammalian, including human, primary cells isolated from brain tissue, for example, or established cell lines, and/or transfected or transformed cell cultures, such as those available from the American Type Culture Collection (ATCC), 10801 University Boulevard, Manassas, Va. 20110-2209.

Test compounds employed in the screening methods of this invention are as described herein, and include for example, without limitation, synthetic organic compounds, chemical compounds, natural products, polypeptides and peptides.

In addition, the screening methods of the present invention allow for efficient cell-based screening of inhibitor compounds, and in particular, of inhibitors of a critical proteolytic event in the generation of Aβ associated with Alzheimer's disease. The screening and detection methods of the present invention have been demonstrated in a nonlimiting manner using the compound 3-methyladenine (3MA), (see, Example 6 and FIGS. 10A-10D), which specifically reduces the levels of the APP metabolite βCTF, which is involved in a metabolic pathway associated with AD progression.

Further, the methods of this invention, in which the levels of both a metabolic precursor and a biologically meaningful metabolite are both determined, have applicability in high-throughput screens for other cell-based assays. Illustratively, the present invention contemplates methods for screening for a specific decrease or increase in phospho-epitopes on tau that are relevant to the pathological accumulation of paired helical filaments. For example, in such a condition, the metabolic precursor is tau and the biologically meaningful metabolite is a specific phosphorylated form of tau (i.e., phospho-tau).

In addition, the detection of compounds that allow an increase in a protein or peptide, for example, the APP precursor, or one or more particular metabolites, e.g., secreted APP, may be beneficial. For example, secreted APP may have neuroprotective effects. Further, generation of the αCTF precludes Aβ generation and therefore can have a protective effect.

Accordingly, in one of its aspects, the present invention embraces cell-based screening or detection methods to identify compounds that modulate (i.e., (i) reduce, inhibit, decrease, or block; or (ii) increase, enhance, or augment) the generation of one or more cellular metabolites associated with a disease or disorder, without causing non-specific cytotoxicity. The methods involve contacting cells with a test compound; determining the levels of a cellular precursor protein, or the levels of a precursor conformation state of a cellular protein; and then determining levels of a metabolite generated from the cellular precursor protein. The metabolite can be a breakdown or cleavage product of the precursor protein, or it can be a modified form of the state of the precursor polypeptide, e.g., a post-translationally modified polypeptide. According to the method, a test compound that specifically reduces or inhibits the level of the metabolite in cells shows a reduction only in the level of the metabolite relative to that in untreated cells, and a test compound that is non-specifically cytotoxic shows a reduction in the levels of both the cellular precursor protein, and, optionally, its associated cleavage products and the metabolite.

In another of its aspects, the present invention embraces a cell-based screening or detection method to identify compounds that increase or augment the generation of one or more cellular metabolites associated with a decreased likelihood of developing, or decreasing the severity of, a disease or disorder, without causing non-specific cytotoxicity. These methods involve contacting cells with a test compound; determining levels of a cellular precursor protein or the levels of a precursor conformation state of a cellular protein; and then determining levels of a metabolite generated from the cellular precursor protein. The metabolite is as described above. In accordance with this method, a compound that specifically increases or augments the level of the metabolite in cells shows an increase in the levels of the metabolite relative to untreated cells, while not significantly altering the levels of the precursor.

Thus, the present invention provides a broad cell-based screening approach that is suitable for use with many drug identification and discovery schemes, preferably in a high-throughput screening format. Automated high throughput screening is-described, for example, in Burbaum et al., 1997, Current Opinion in Chemical Biology, 1:72-78; and Schullek et al., 1997, Analyt. Biochem., 246:20-29. As a nonlimiting example, in high-throughput screening according to the present invention, liquid handling operations can be performed by a Microlab 2000.™. pipetting station (e.g., Hamilton Instruments). Other equipment needed for the screening (e.g. incubators, plate washers, plate readers) can either be adapted for automated functioning, as necessary, or commercially purchased as automated modules. Movement of samples through the assay can be performed by robots, for example, an XP.™. robot mounted on a 3 m-long track (Zymark, Hopkinton, Mass.)

Through the screening method according to this invention, libraries of synthetic organic compounds, natural products, peptides, and oligonucleotides can be evaluated for their capacity to modulate particular target metabolic proteins and their products (e.g., cleavage products) that reflect or contribute to a disease process. Specifically, compounds can be identified that target components in a metabolic pathway associated with a disease or disorder, e.g., a metabolic precursor or a proteolytic enzyme involved in the processing of the precursor. For example, compounds can be detected or identified that specifically inhibit β-secretase proteolytic activity on APP so as to prevent conversion to, or accumulation of, resulting cellular metabolites that can cause or exacerbate a disease.

In yet another of its aspects, the present invention encompasses an active compound or compounds tested in, or identified or detected from, the performance of the methods as, described herein. A nonlimiting, representative compound that demonstrated efficacy in the methods of the invention (Example 6) is 3-methyladenine (3MA) and related derivative, analog, or modified forms thereof that preferably do not alter its function or activity.

Active compounds can be optimized, if desired, via medicinal chemistry. Initially, for example, pharmacophore(s) can be defined using modern computational chemistry tools and that are representative of the structures found to be active in the high throughput screens. Once at consensus pharmacophore is identified, focused combinatorial libraries of compounds can be designed to probe structure-activity relationships. Finally, the biopharmaceutical properties, such as potency and efficacy, of a set of lead structures can be improved to identify suitable compounds for clinical testing.

Thus, the present invention provides novel cell-based methods for identifying compounds that can be utilized in therapeutic methods for treating diseases and conditions resulting from an intracellular precursor-product activity that causes the production, or buildup, of product leading to disease or augmentation of disease.

Antibodies and Antibody Production

The term antibody refers to intact molecules as well as fragments thereof, such as Fab, F(ab′0 )₂, Fv, which are capable of binding an epitopic or antigenic determinant. Antibodies that bind to a metabolic protein, polypeptide or peptide, e.g., APP, can be prepared using intact polypeptides or fragments containing small peptides of interest or prepared recombinantly for use as the immunizing antigen. (see, Table 1, Example 1). The polypeptide or oligopeptide used to immunize an animal can be derived from the translation of RNA or synthesized chemically, and can be conjugated to a carrier protein, if desired. Commonly used carriers that are chemically coupled to peptides include bovine serum albumin (BSA), keyhole limpet hemocyanin (KLH), and thyroglobulin. The coupled peptide is then used to immunize the animal (e.g, a mouse, rat, sheep, goat, or rabbit).

The term “humanized” antibody refers to antibody molecules in which amino acids have been replaced in the non-antigen binding regions, e.g., the complementarity determining regions (CDRs), in order to more closely resemble a human antibody, while still retaining the original binding capability, e.g., as described in U.S. Pat. No. 5,585,089 to C. L. Queen et al., which is a nonlimiting example. Fully humanized antibodies, such as those produced transgenically or recombinantly, are also encompassed herein.

The term “antigenic determinant” refers to that portion of a molecule that makes contact with a particular antibody (i.e., an epitope). When a protein or fragment of a protein is used to immunize a host animal, numerous regions of the protein may induce the production of antibodies which bind specifically to a given region or three-dimensional structure on the protein; these regions or structures are referred to as antigenic determinants. An antigenic determinant may compete with the intact antigen (i.e., the immunogen used to elicit the immune response) for binding to an antibody.

The terms “specific binding” or “specifically binding” refer to the interaction between a protein or peptide and a binding molecule, such as an agonist, an antagonist, or an antibody. The interaction is dependent upon the presence of a particular structure (e.g., an antigenic determinant or epitope, or a structural determinant) of the protein that is recognized by the binding molecule. For example, if an antibody is specific for epitope “A”, the presence of a protein containing epitope A (or free, unlabeled A) in a reaction containing labeled “A” and the antibody will reduce the amount of labeled A bound to the antibody.

Antibodies specific for a metabolic precursor polypeptide, e.g., APP, or metabolic product, e.g., βCTF or Aβ, or immunogenic peptide fragments thereof, can be generated using methods that have long been known and conventionally practiced in the art. Such antibodies may include, but are not limited to, polyclonal, monoclonal, chimeric, single chain, Fab fragments, and fragments produced by an Fab expression library. Neutralizing antibodies, (i.e., those which inhibit dimer formation) are especially preferred for therapeutic use.

For the production of antibodies, various hosts including goats, rabbits, sheep, rats, mice, humans, and others, can be immunized by injection with the appropriate polypeptide, or any peptide fragment or oligopeptide thereof, which has immunogenic properties. Depending on the host species, various adjuvants may be used to increase the immunological response. Nonlimiting examples of suitable adjuvants include Freund's (incomplete), mineral gels such as aluminum hydroxide or silica, and surface active substances such as lysolecithin, pluronic polyols, polyanions, peptides, oil emulsions, KLH, and dinitrophenol. Adjuvants typically used in humans include BCG (bacilli Calmette Guérin) and Corynebacterium parvumn.

Preferably, the peptides, fragments, or oligopeptides used to induce antibodies to the polypeptides (i.e., immunogens) have an amino acid sequence having at least five amino acids, and more preferably at least 6 to 10 amino acids. It is also preferable that the immunogens are identical to a portion of the amino acid sequence of the natural protein; they may also contain the entire amino acid sequence of a small, naturally occurring molecule. The peptides, fragments or oligopeptides may comprise a single epitope or antigenic determinant or multiple epitopes. Short stretches of amino acids comprising the protein or peptide can be fused to those of another protein, such as KLH, in which case antibodies can be produced against the chimeric molecule.

Monoclonal antibodies to metabolic precursor proteins and metabolite product polypeptides, peptides, or immunogenic fragments thereof, may be prepared using any technique which provides for the production of specific antibody molecules by continuous cell lines in culture. These include, but are not limited to, the hybridoma technique, the human B-cell hybridoma technique, and the EBV-hybridoma technique (G. Kohler et al., 1975, Nature, 256:495-497; D. Kozbor et al., 1985, J. Immunol. Methods, 81:31-42; R. J. Cote et al., 1983, Proc. Natl. Acad. Sci. USA, 80:2026-2030; and S. P. Cole et al., 1984, Mol. Cell Biol., 62:109-120). The production and screening of monoclonal antibodies is well known and routinely used in the art.

In addition, techniques developed for the production of “chimeric antibodies,” the splicing of mouse antibody genes to human antibody genes to obtain a molecule with appropriate antigen specificity and biological activity can be used (S. L. Morrison et al., 1984, Proc. Natl. Acad. Sci. USA, 81:6851-6855; M. S. Neuberger et al., 1984, Nature, 312:604-608; and S. Takeda et al., 1985, Nature, 314:452-454). Alternatively, techniques described for the production of single chain antibodies may be adapted, using methods known in the art, to produce single chain antibodies specific for a particular protein or peptide. Antibodies with related specificity, but of distinct idiotypic composition, may be generated by chain shuffling from random combinatorial immunoglobulin libraries (D. R. Burton, 1991, Proc. Natl. Acad. Sci. USA, 88:11120-3). Antibodies may also be produced by inducing in vivo production in a lymphocytic cell population or by screening recombinant immunoglobulin libraries or panels of highly specific binding reagents as disclosed in the literature (R. Orlandi et al., 1989, Proc. Natl. Acad. Sci. USA, 86:3833-3837 and G. Winter et al., 1991, Nature, 349:293-299).

Antibody fragments which contain specific binding sites for, a given protein or peptide may also be generated. For example, such fragments include, but are not limited to, F(ab′)₂ fragments which can be produced by pepsin digestion of the antibody molecule and Fab fragments which can be generated by reducing the disulfide bridges of the F(ab′)₂ fragments. Alternatively, Fab expression libraries may be constructed to allow rapid and easy identification of monoclonal Fab fragments with the desired specificity (W. D. Huse et al., 1989, Science, 254.1275-1281).

Various immunoassays can be used for screening to identify antibodies having the desired specificity. Numerous protocols for competitive binding or immunoradiometric assays using either polyclonal or monoclonal antibodies with established specificities are well known in the art. Such immunoassays typically involve measuring the formation of complexes between a particular protein or polypeptide and its specific antibody.

Screening Methods

A variety of protocols for detecting and measuring proteins and peptides using either polyclonal or monoclonal antibodies specific for the protein or peptide are known and practiced in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive with two non-interfering epitopes on a polypeptide can be employed, as can competitive binding assays. These and other assays are described in the art, as represented by the publications of R. Hampton et al., 1990; Serological Methods, a Laboratory Manual, APS Press, St Paul, Minn. and D. E. Maddox et al., 1983; J. Exp. Med., 158:1211-1216).

The novel cell-based screening assays described herein can be used to identify candidate bioactive agents or drugs that modulate, preferably reduce or inhibit, the function or bioactivity of a metabolic precursor. In this way agents can be identified for use in treating diseases and disorders characterized by the production of a proteolytic breakdown product, for example, so that cells harboring the target precursor protein (e.g., a proteolytic enzyme) can be killed or growth arrested.

Generally, in performing screening methods, cells, or even polypeptides or peptides involved in a particular metabolic pathway, can be non-diffusably bound to an insoluble support having isolated sample receiving areas (e.g. a microtiter plate, an array, etc.). The criteria for suitable insoluble supports are that they can be made of any composition to which cells or polypeptides can be bound; they are readily separated from soluble material; and they are otherwise compatible with the overall method of screening. The surface of such supports may be solid or porous and of any convenient size or shape. Examples of suitable insoluble supports include microtiter plates, arrays, membranes and beads. These are typically made of glass, plastic (e.g., polystyrene), polysaccharides, nylon or nitrocellulose.

Microtiter plates and arrays are especially convenient, because a large number of assays can be carried out simultaneously, using small amounts of reagents and samples. The particular manner of binding the polypeptide is not crucial, so long as it is compatible with the reagents and overall methods of the invention, maintains cell viability or the activity of the peptide and is nondiffusable.

Preferred methods of binding include the use of antibodies, direct binding to “sticky” or ionic supports, chemical crosslinking, etc. Following binding of the cells or polypeptides, excess unbound material is removed by washing. The sample receiving areas may then be blocked as needed through incubation with, for example, bovine serum albumin (BSA), casein or other innocuous/nonreactive protein.

A candidate bioactive agent or drug is added to the assay. Novel binding agents can include specific antibodies, non-natural binding agents identified in screens of chemical libraries, peptide analogs, etc. Of particular interest are screening assays for agents that have a low toxicity or human cells; however, in accordance with the present invention, agents that are normally toxic to cells can be successfully assayed in the cell-based methods as described herein.

ELISA immunoassays are preferred for identifying suitable; drugs or bioactive agents according to the present invention. In addition other assays can be used for this purpose, including labeled in vitro protein-protein binding assays, electrophoretic mobility shift assays, other immunoassays for protein binding, and the like. The term “agent” as used herein refers to any molecule, e.g., protein, oligopeptide, small organic molecule, polysaccharide, polynucleotide, etc., having the capability off directly or indirectly altering the activity or function of a target molecule, such as a metabolic precursor polypeptide. Generally a plurality of assay mixtures are run in parallel with different agent concentrations to obtain a differential response to the various concentrations. Typically, one of these concentrations serves as a negative control, i.e., at zero concentration, or below the level of detection.

Candidate agents, compounds, drugs, and the like encompass numerous chemical classes, though typically they are organic molecules, preferably small organic compounds having a molecular weight of more than 100 and less than about 10,000 daltons, preferably, less than about 2000 to 5000 daltons, as a nonlimiting example. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures,and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate bioactive agents, compounds, drugs, biomolecules and the like are obtained from a wide variety of sources, including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. In addition, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

A variety of other reagents may be included in the screening assay according to the present invention. Such reagents include, but are not limited to, salts, neutral proteins, e.g. albumin, detergents, etc., which may be used to facilitate optimal protein-protein binding and/or to reduce non-specific or background interactions. In addition, reagents that otherwise improve the efficiency of the assay, such as protease inhibitors, anti-microbial agents, etc. may be used. Further, the mixture of components in the method may be added in any order that provides for the requisite binding.

Kits are included as an embodiment of the present invention which comprise containers with reagents necessary to screen test compounds. Depending on the design of the test and the types of compounds to be screened, such kits include antibodies to metabolic precursor polypeptide, or peptide, and/or antibodies to metabolite product, labeled or unlabeled, and instructions for performing the assay.

### EXAMPLES

The following examples describe specific aspects of the invention to illustrate the invention and provide a description of the present methods for those of skill in the art. The examples should not be construed as limiting the invention, as the examples merely provide specific methodology useful in understanding and practice of the invention and its various aspects.

### Example 1

Cell lines, cDNA constructs, and transfections. Ltk-cells (a murine fibroblast-like cell line; (S. Kit et al., 1967, J. Virol., 1(1): 238-240) were maintained at 37° C. and 5% CO₂ in high glucose DMEM, (Celigro, Hedon, Va.) supplemented to contain 10% fetal bovine serum (Gemini, Woodland, Calif.), 2 mM glutamax I (Gibco/BRL, Gaithersburg, Md.), and penicillin/streptomycin (Celigro). cDNAs encoding human APP₆₉₅ and human rab5 were inserted into the mammalian expression vector pcDNA3 (Invitrogen, Carlsbad, Calif.). Following transfection using lipofectin (manufacturer's protocol; Gibco/BRL), stable L cell lines overexpressing APP₆₉₅ were selected in 400 μg/ml G418 (Gemini) and screened for expression. Transient transfections with rab5 were done using lipofectAMINE (Gibco/BRL) or fugene (Boehringer-Mannheim, Indianapolis, Ind.) according to the manufacturers' protocol.

Antibodies. Table I describes the monoclonal antibodies used in the methods according to the present invention.

TABLE I
Monoclonal antibodies

| | Epitope | APP-species | Immonogen |
|---|---|---|---|
| Antibody | Specificity | detected | Sequence |
| C1/6.1 | within carboxy- | APP holopro- | KMQQNGYENP |
| | terminal 20 | tein; all CTFs | TYKFFEQMQN |
| | residues of APP | | (SEQ ID NO:2) |
| C2/7.1 | within residues | APP holopro- | LVMLKKKQYTS |
| | 644-676 of | tein; all CTFs | IHHGVVEVDAA |
| | APP₆₉₅ | | VTPEERHLSK |
| | | | (SEQ ID NO:3) |
| JRF/AβN/25 | within first 7 | βCTF; | DAEFRHD |
| | residues of Aβ; | Aβ1-X by | (SEQ ID NO:4) |
| | requires β- | ELISA | |
| | cleavage | | |
| JRF/Aβtot/17 | within residues | APP holopro- | DAEFRHDSGY |
| | 1-16 of | tein; βCTF; | EVHHQKLVFFA |
| | human Aβ | Aβ by ELISA | EDVGSNKGAII |
| | | | GLMVGGVV |
| | | | (SEQ ID NO:5) |

Table 1 shows the monoclonal antibodies and the known epitope specificity based upon the immunogenic peptide used for immunization, as well as the antibody's binding by ELISA to additional synthetic peptides. Additionally, the binding of these antibodies to various APP proteolytic species is as described herein. The specificity of JRF/AβN/25 and JRF/Aβtot/17 for Aβ, and the use of these two monoclonal antibodies in an Aβ sandwich ELISAs is as described (C. Janus et al., 2000, Nature, 408(6815): 979-982; S.D. Schmidt et al., 2001, Society for Neuroscience, annual meeting 2001).

The C1/6.1 antibody was raised against the conserved carboxy-terminal 20 residues of APP (residues 676-695 of APP₆₉₅), (SEQ ID NO:2) and is useful for immunolabeling, immunoprecipitation, and Western blot analysis (see also, C. Janus et al., 2000, Nature, 408(6815):979-982). The C2/7.1 antibody was raised against residues 644-676 of APP₆₉₅ (SEQ ID NO:3), and is also useful for immunolabeling, immunoprecipitation, and Western blot analysis. JRF/AβN/25 was raised against a synthetic peptide encompassing residues 1 to 7 of human Aβ. Extensive evidence that JRF/AβN/25 requires β-cleavage at residue 1 of Aβ is presented herein. Other antibodies, their specificity and their use in a sandwich ELISA are as described. (C. Janus et al., Id.)

Sandwich ELISAs. Sandwich ELISAs using the antibodies as presented in Table 1 were performed essentially as has been reported (C. Janus et al. Id.; R. Rozmahel et al., Neurobiology of Aging, 23:187-194; and P. M. Mathews et al., 2002, J. Biol. Chem., 277: 5299-5307), with modifications to detect cell-associated APP metabolites. The wells of Nunc-Immuno PIates (Nunc A/S, Roskilde, Denmark) were coated overnight at 4° C. using 20 μg/ml of C1/6.1 and the remaining protein binding sites were blocked by incubating with 1% Block Ace (Yukijirushi Milk, Sapporo Japan) in PBS (200 μl/well) for 4 hours at room temperature.

For the βCTF ELISA, a synthetic peptide was prepared, i.e., DAEFRHDKMQQNGYENPTYKFFEQMQN, (SEQ ID NO:1), that contains both the JRF/AβN/25 epitope (SEQ ID NO:4) and the C1/6.1 epitope (SEQ ID NO:2), (see Table I). For the APP/total CTF ELISA, a glutathione S-transferase fusion protein containing the C-terminal 99 residues of human APP (GST-βPP672-770; (K. Islam and E. Levy, 1997, Am. J. Pathol. 151(1):265-71) was prepared. This fusion protein contains both the C1/6.1 and C2/7.1 epitopes. These standards, prepared as stock solutions, were dissolved in DMSO, stored at −70° C., and were further diluted in buffer containing 20 mM Na phosphate, 2 mM EDTA,. 400 mM NaCl, 0.2% BSA, 0.05% CHAPS, 0.4% Block Ace and 0.05% NaN₃, pH 7.0 immediately, prior to use.

ELISA plates were incubated overnight at 4° C. with samples and standards. Samples, as described herein, were cell lysates prepared in 1% Triton X-100®, 140 mM NaCl, 25 mM Tris (pH 7.4), 0.5 mM EDTA, and protease inhibitors. Lysates were vortexed briefly, allowed to rest on ice for 30 minutes, and centrifuged at 6,000 rpm in an Eppendorf centrifuge. The supernatant was used for the ELISA. Following overnight binding of APP and APP metabolites to the capture antibody (C/1/6.1), the wells were washed twice with phosphate buffered saline (PBS) containing 0.5% Triton X-100®/0.05% Tween-20 followed by two washes with PBS. A solution containing 20 mM Na phosphate, 2 mM EDTA, 400 mM NaCl, and 1% BSA, pH 7.0 was then added to the wells for 1 hour at room temperature.

APP and cell-associated APP metabolites were detected by incubating for 4 hours at room temperature with horseradish peroxidase (HRP)-conjugated C2/7.1 or JRF/AβN/25 diluted in 20 mM Na phosphate, 2 mM EDTA, 400 mM NaCl, 1% BSA, pH 7.0. Thereafter, the wells were again washed twice with phosphate buffered saline (PBS) containing 0.5% Triton X-100®/0.05% Tween-20, followed by two washes with PBS. ELISA plates were developed using a color reaction (TMB Microwell Peroxidase Substrate System, Kirkegaard & Perry, Gaithersburg, MD) and the OD₄₅₀ was read. ELISA signals are reported as the mean ±SE of two or more wells in femtomoles per ml relative to standard.

Metabolic labeling, immunoprecipitation and Western blot analysis. To detect APP and CTFs, 5×10⁵ cells were seeded onto 35-mm diameter tissue culture dishes followed by induction of human APP₆₉₅ expression with 20 mM butyrate for 24 hours (P. M. Mathews et al., 1992, J. Cell. Biol., 118(5):1027-1040; P. M. Mathews et al., 2000 Mol Med., 6(10)878-891). Cultures were methionine/cysteine starved for 20 minutes, pulse-labeled for 15 minutes with 100 μCi/ml TRANS³⁵S-LABEL (Dupont-NEN), washed, and chased in complete medium containing 2 mM unlabeled methionine (P. M. Mathews et al., 1992, J. Cell. Biol., 118(5):1027-1040). Calpain inhibitors, as described herein, were added to the growth medium 3 hours prior to methionine/cysteine starvation and then throughout pulse-labeling and chase.

Cell lysates (prepared in 1% Triton X-100, 140 mM NaCl, 25 mM Tris pH 7.4, 0.5 mM EDTA, 10 mM methionine and protease inhibitors; (P. M. Mathews et al., 1992, J. Cell. Biol., 118(5):1027-1040; A. Beggan et al., 1996, J. Biol. Chem., 271(34):20895-20902) were subjected to immunoprecipitation with various monoclonal antibodies as described in Example 2. Immunoprecipitated proteins were sized by SDS-PAGE and labeled proteins were visualized by exposure to x-ray film and analyzed quantitatively using a Storm 840 phosphorimager (Molecular Dynamics/Amersham Biosciences, Sunnyvale, Calif. and/or by scan analysis (NIH Image).

For Western blot analysis, protein concentration in cell lysates was determined (BioRad DC Protein Assay, BioRad, Rockville Center, N.Y.), and equal amounts of proteins were separated by SDS-PAGE and transferred to PVDF membrane. Membranes were incubated in C1/6.1 (2 μg/ml) overnight, washed, and incubated with HRP-conjugated goat anti-mouse IgG for 1.5 hours. Membranes were incubated in ECL substrate (Amersham, Arlington Heights, Ill.) and exposed to x-ray film.

Preparation of cell lysates for ELISA. Cells were seeded into 6-well dishes and allowed to settle overnight. Human APP₆₉₅ expression in L cells was induced with 20 mM butyrate for approximately 40 hours (P. M. Mathews et al., 1992, J. Cell. Biol., 118(5):1027-1040), cells were washed twice with Hank's BSS before extraction in 0.5 ml lysis buffer (1% Triton X-100, 140 mM NaCl, 25 mM Tris pH 7.4, 0.5 mM EDTA, protease inhibitors). Cell lysates were vortexed briefly, allowed to rest on ice for approximately 15 minutes, and gently spun at 6,000 rpm in an Eppendorf centrifuge. Supernatants were collected and loaded neat for ELISA.

### Example 2

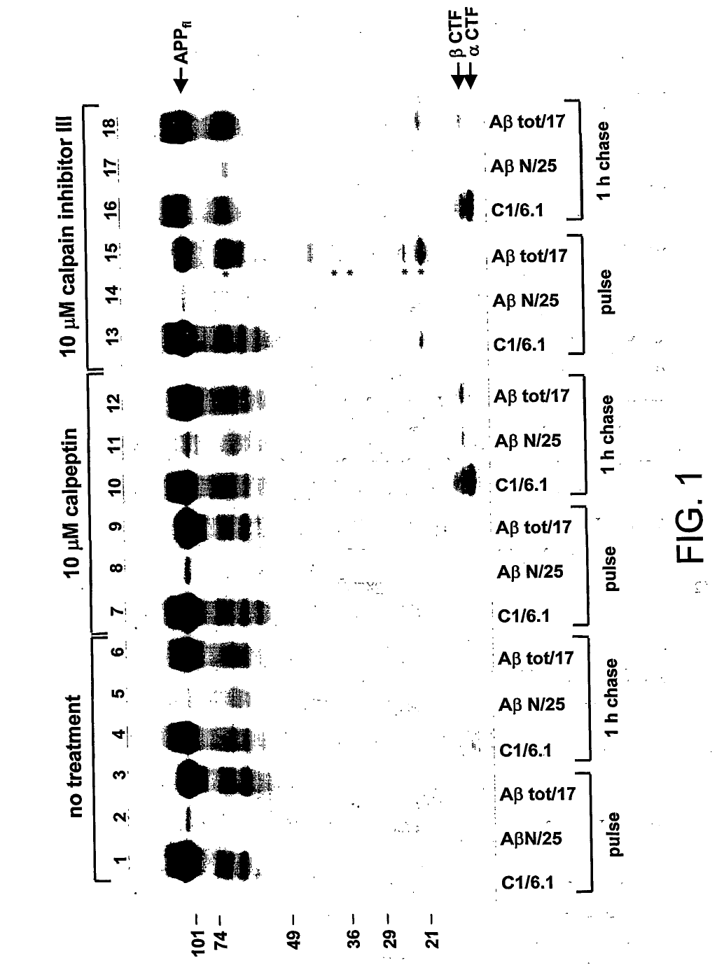

Specificity of monoclonal antibodies. Table I in Example 1 presents the monoclonal antibodies used in the method according to the invention, as well as the sequences of the synthetic peptides used to generate each antibody. APP holoprotein and CTFs were immunoprecipitated using monoclonal antibodies that recognize different epitopes within the carboxy-terminal 99 residues of APP: C1/6.1, JRF/AβN/25, and JRF/Aβtot/17 (see Table I and FIG. 1).

To determine whether a particular monoclonal antibody detected APP holoprotein and/or αCTF and/or βCTF or both, L cells overexpressing human APP₆₉₅ (L/APP cells) were metabolically labeled for 15 minutes, and then were chased for 1 hour (FIG. 1, lanes 1-6).

Calpain inhibitors are known to increase the levels of APP CTFs within cells (H. Klafki et al., 1996, J. Biol. Chem., 271(45):28655-28659; L. Zhang et al., 1999, J. Biol. Chem., 274(13):8966-9872; G. Verdile et al., 2000, J. Biol. Chem., 275(27):20794-20798). It has recently been reported that, contrary to previous reports, inhibiting calpains increases the levels of CTFs by increasing the biosynthesis of both α- and β-cleaved CTFs (P. M. Mathews et al., 2001, Society for Neuroscience, annual meeting 2001).

Thus, in addition to control conditions, cells were treated for 3 hours prior to, as well as during, the pulse and chase periods with the indicated calpain inhibitors (FIG. 1, lanes 7-18) to increase the levels of metabolically labeled CTFs prior to immunoprecipitation. Equal volumes of detergent lysates prepared from the pulse and chase periods were subjected to immunoprecipitation with each of the monoclonal antibodies and labeled APP species were resolved on 4-20% gradient SDS-PAGE.

In untreated L/APP cells (FIG. 1, lanes 1-6), C1/6.1 immunoprecipitated labeled holoprotein from the pulse period (FIG. 1, lane 1). Following the 1 hour chase, C1/6.1 immunoprecipitation also revealed two rapidly migrating APP fragments (FIG. 1, lane 4; arrows indicating αCTF and βCTF). JRF/AβN/25 immunoprecipitation failed to bring down APP holoprotein (FIG. 1, lanes 2 and 5), thus confirming that the epitope for JRF/AβN/25 requires a cleaved amino-terminus at residue 1 of Aβ. That JRF/AβN/25 immunoprecipitated, following the 1 hour chase, a protein that co-migrated with one of the CTFs revealed by C1/6.1 immunoprecipitation (compare FIG. 1, lanes 4 and 5), conclusively identified this as the β-cleaved CTF of APP.

JRF/Aβtot/17, which recognizes an epitope that resides within residues 1-16 of Aβ, immunoprecipitated APP holoprotein in the pulse and chase periods (FIG. 1, lanes 3 and 6, respectively), as well as the βCTF following chase. Given the specificities of these three monoclonal antibodies and the mobility on SDS-PAGE of the CTFs that they immunoprecipitated, it was concluded that the most rapidly migrating species identified by C1/6.1 was the α-cleaved CTF.

In cells treated with either 10 μM calpeptin or 10 μM calpain inhibitor III, the immunoprecipitation pattern from the pulse-labeled lysates was similar to that seen in untreated cells (compare FIG. 1, lanes 1-3 with lanes 7-9 and lanes 13-15). However, with calpain inhibition, striking differences in the abundance of CTFs were seen following the 1 hour chase. C1/6.1 immunoprecipitation demonstrated a substantial and apparently proportionate increase in the levels of both αCTFs and βCTFs with either calpain inhibitor (compare FIG. 1, lane 4 with lanes 10 and 16). This increase in the βCTF was confirmed by immunoprecipitation with JRF/AβN/25 and JRF/Aβtot/17 (FIG. 1, lanes 11, 12, 17 and 18).

Densitometric quantification of these bands from the C1/6.1 immunoprecipitation confirmed the observation described herein that the ratio of αCTF to βCTF appeared not to be affected by calpain inhibition (remaining at approximately 2:1), in spite of an approximate 4-fold increase in the biosynthesis of both QTFs. Finally, it is noted that the APP holoprotein contains 5 times more methionines than do the CTFs, thus, the metabolic labeling substantially under-estimates the relative abundance of the CTFs.

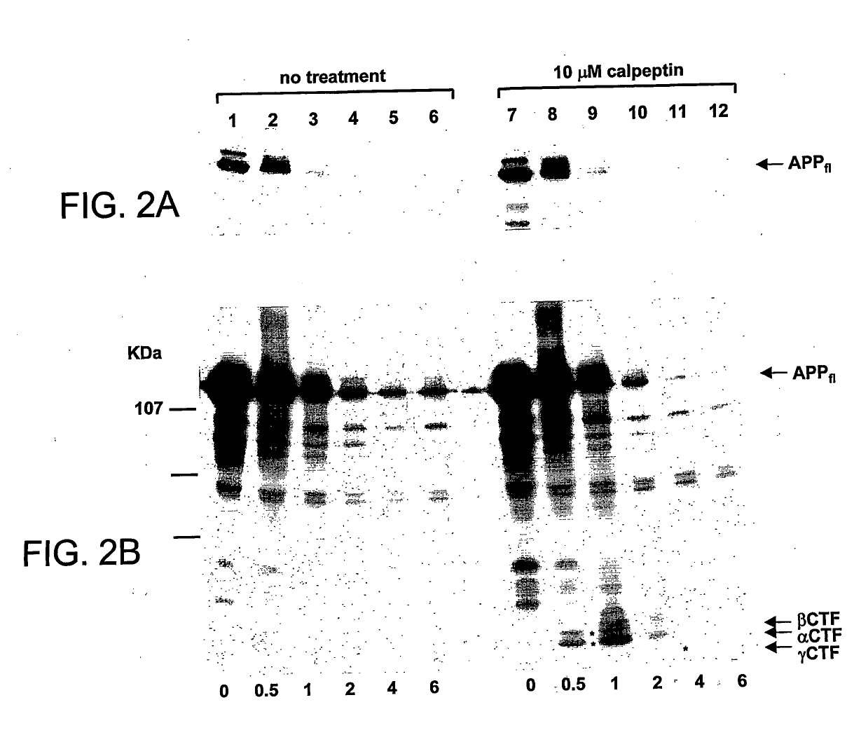

The pulse-chase experiment shown in FIGS. 2A and 2B was designed to determine if C1/6.1 was able to detect other species of CTFs (e.g. the γCTF). Accordingly, L/APP cells were pulse-labeled for 15 minutes and chased for the indicated times up to 6 hours prior to immunoprecipitation of lysates with C1/6.1. FIG. 2A shows that the turnover of APP holoprotein in control (lanes 1-6) and in 10 μM calpeptin treated cells (lanes 7-12) is similar. Quantification of these data confirmed that the turnover of APP holoprotein was unchanged by calpeptin treatment.

FIG. 2B shows a longer exposure of the same immunoprecipitation revealing the generation and turnover of the CTFs. In agreement with the data in FIG. 1, 10 μM calpeptin treatment substantially increased the generation of both α and βCTFs during the initial 1 hour of chase (compare FIG. 2B, lanes 1-3 with lanes 7-9). In contrast to a previous interpretation (L. Zhang et al., 1999, J. Biol. Chem., 274(13):8966-8972), this calpeptin concentration did not appear to reduce the turnover of CTFs. While the generation of CTFs is substantially increased, the rate of their degradation, like the turnover of APP, did not appear to be affected by calpeptin treatment.

In addition, the long exposure shown in FIG. 2B highlights other CTFs that are consistently detected. This includes fragments recognized by C1/6.1 that migrate above βCTF (FIG. 2B, lane 9), as well as a fragment that migrates more rapidly than αCTF and that appears following long chase periods (>2 hours), (FIG. 2B, lanes 10-12, labeled γCTF). The larger fragments suggest cleavage heterogeneity amino-terminal to the β-cleavage site. The fragment smaller than the αCTF is consistent in size and time course of appearance with the γCTF of APP.

Thus, it was concluded from the results in Example 2 that C1/6.1 antibody detected all of the known cell-associated metabolites of APP (APP holoprotein and CTFs) and that JRF/AβN/25 antibody specifically detected the β-cleaved CTF. Also, since the C2/7.1 antibody was raised against the carboxy-terminus of APP, as was C1/6.1, and results using the C2/7.1 monoclonal antibody were similar to those obtained using C1/6.1, it was concluded that C2/7.1 also detects APP holoprotein and all CTFs.

### Example 3

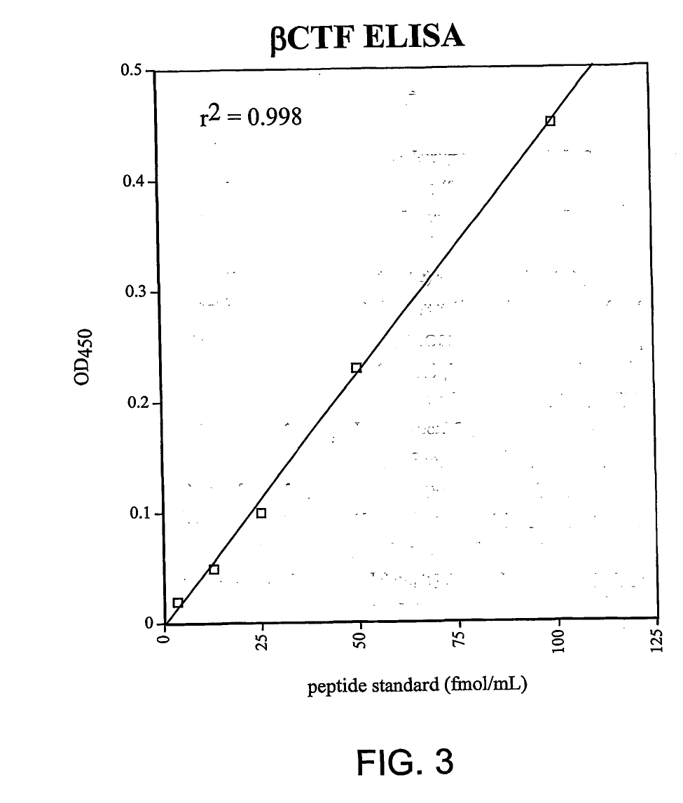

The βCTF specific ELISA. Using C1/6.1 as the capture antibody and JRF/AβN/25 as the detection antibody, an ELISA was developed that specifically recognizes cell-associated βCTFs. FIG. 3 illustrates the sensitivity and linearity of this ELISA against a synthetic peptide (DAEFRHDKMQQNGYENPTYKFFEQMQN), (SEQ ID NO:1) containing the JRF/AβN/25 epitope (SEQ ID NO:4) at its amino-terminus (in bold) and the C1/6.1 epitope (SEQ ID NO:2) at its carboxy-terminus (in italics).

This FIG. 3 ELISA shows linear detection into the low fmole/ml range (range shown is 3 to 100 fmole/ml), similar to the range that was obtained with Aβ sandwich ELISAs (C. Janus et al., 2000, Nature, 408(6815):979-982; S. D. Schmidt et al., 2001, Society for Neuroscience, annual meeting 2001) and well within the range necessary to detect the βCTFs generated in vivo by a cell.

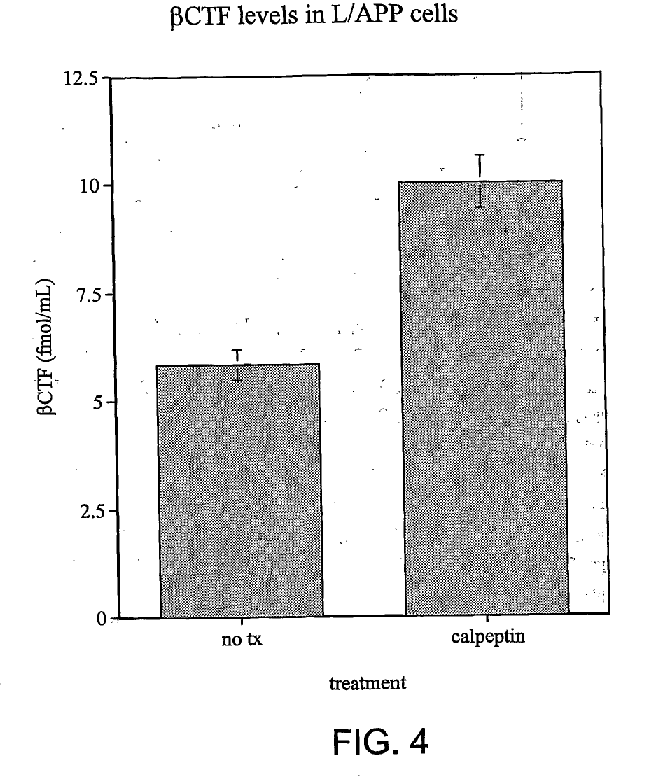

To determine whether such an ELISA protocol could be used to detect βCTFs isolated from cells, detergent lysates prepared from equal density L/APP cells and L/APP cells treated with 10 μM calpeptin for 6 hours were examined. In untreated L/APP cells, 5.8±0.3 fmole/ml of βCTFs were detected (FIG. 4). The addition of 10 μM calpeptin for 6 hours nearly doubled the levels of βCTFs detected by ELISA (10.0±0.6 fmole/ml), in agreement with pulse-labeling data showing an increase in βCTFs following calpeptin treatment. This result demonstrates that the βCTF ELISA can quantitatively detect changes in levels of βCTFs generated within cells.

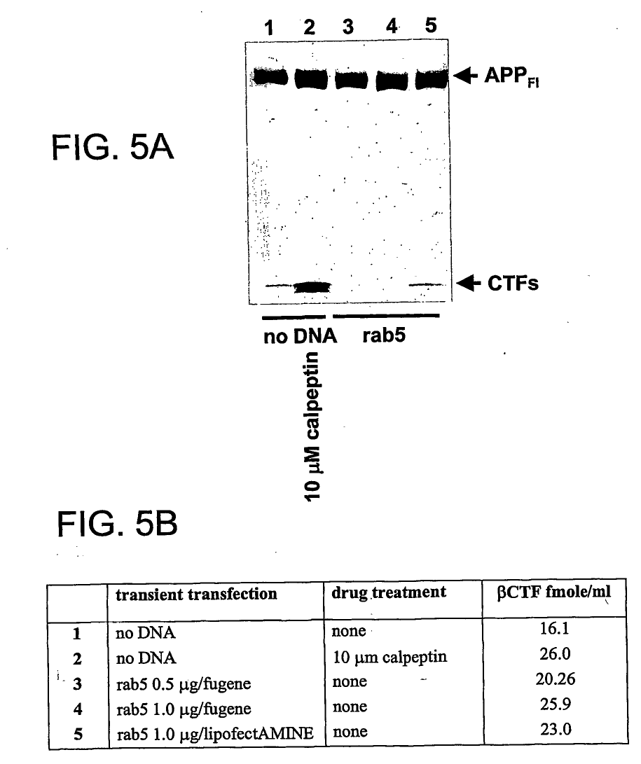

Additional results shown in FIGS. 5A and 5B confirm that this βCTF ELISA can be used to detect changes in the amount of βCTFs generated by a cell. Abnormalities of the neuronal endosomal system seen in early-stage, sporadic Alzheimer's disease (see, e.g., A. M. Cataldo et al., 1997, J. Neurosci., 17(16): 6142-6151.(1998); A. M. Cataldo et al., 2000, Am. J. Pathol., 157(1):277-286; R. A. Nixon et al., 2000, Neurochem. Res., 25(9-10):1161-1172) were modeled by overexpressing rab5, an important regulator protein of endocytosis (P. Chavrier et al., 1990, Cell, 62(2):317-329.; J. P. Gorvel et al., 1991, Cell, 64(5):915-925; C. Bucci et al., 1992, Cell, 70(5):715-728; M. A. Barbieri et al., 1996, Biocell, 20(3):331-338; and G. Li, 1996, Biocell, 20(3):325-330; and in patent application U.S. Ser. No.: 09/560,124, entitled, “Methods for the identification of compounds for the treatment of Alzheimer's disease”; to R. A. Nixon et al., filed Apr. 28, 2000).

In this rab5 overexpression experiment, L/APP cells were transiently-transfected with no DNA (FIG. 5A, lanes 1 and 2), or rab5 cDNA (FIG. 5A, lanes 3-5) and the levels of APP holoprotein and CTFs were determined by Western blot analysis using C1/6.1 (FIG. 5A). As expected, treatment with 10 μm calpeptin greatly increased the levels of CTFs detected (compare FIG. 5A, lanes 1 and,2), which the pulse-chase data indicate contain predominantly αCTFs and lesser amounts of βCTFs and γCTFs.

Stimulating endocytosis by rab5 overexpression also increased the levels of CTFs detected by Western blot analysis (most apparent in lane 5, FIG. 5A), although not to the same extent as did calpeptin treatment. In addition to the Western blot analysis, an aliquot of each of the lysates was examined by ELISA to determine the levels of βCTFs in the cells (FIG. 5B). As was seen in FIG. 4, calpeptin treatment substantially increased the amount of βCTFs detected by ELISA (i.e., from 16.1 to 26.0 fmole/ml). In addition, overexpression of rab5 also increased the amount of βCTFs detected by ELISA, nearly to the same extent as did calpeptin treatment, suggesting that the CTFs detected by the C1/6.1 Western blot analysis following rab5 overexpression are predominantly βCTFs, and further supporting the specificity of this ELISA for the βCTF.

These results show that the βCTF ELISA is sensitive, specific, can detect βCTFs generated by a living cell, and can detect changes in the levels of βCTFs resulting from pharmacological as well as genetic, manipulations.

### Example 4

The APP/total CTF ELISA. In addition to the βCTF ELISA, a novel ELISA was developed that detects APP holoprotein, as well as all cell-associated CTFs from cells (APP/total CTF ELISA). This ELISA uses C1/6.1 as the capture antibody, as did the βCTF ELISA, but uses the C2/7.1 antibody, rather than the JRF/AβN/25 antibody, as the detecting antibody.

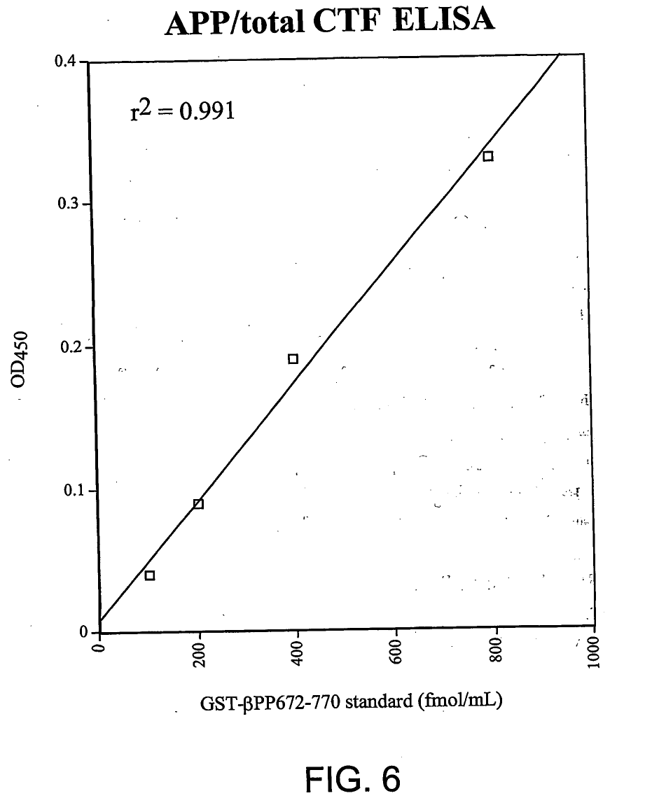

FIG. 6 illustrates the use of this ELISA to detect a standard that consists of the carboxy-terminal 99 amino acids of APP expressed, as a bacterial fusion protein (GST-βPP672-770; (K. Islam and E. Levy, 199 Am. J. Pathol., 151(1):265-271). This fusion protein contains both the C1/6.1 and C2/7.1 epitopes. Like the βCTF ELISA, the APP/total CTF ELISA showed a broad linear range down to low fmole/ml of the standard (as shown in FIG. 6, 100-800 fmole/ml).

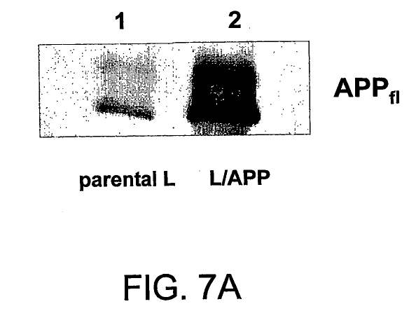

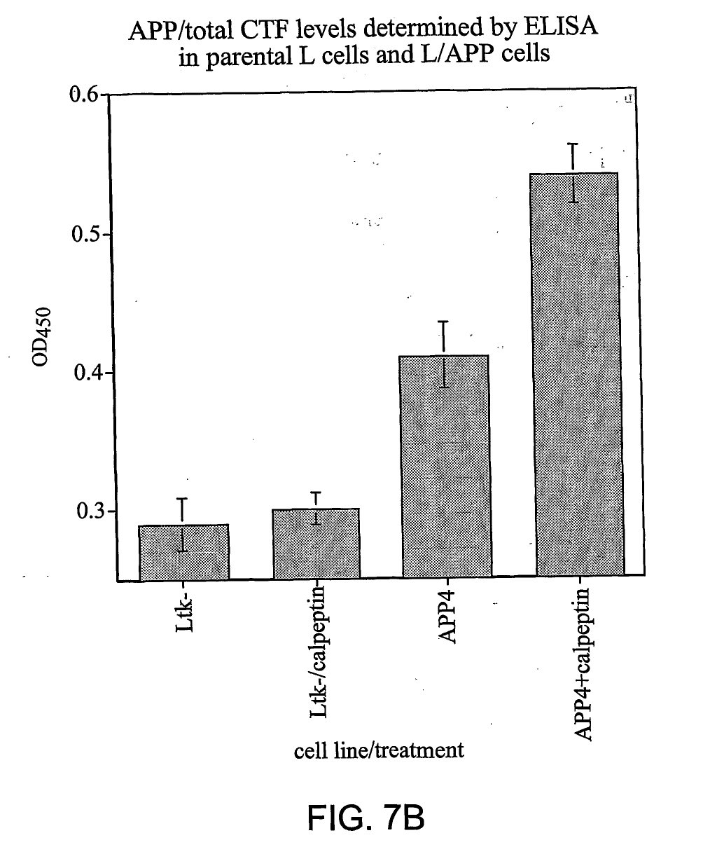

Levels of APP and total CTFs were examined in lysates prepared from cells using this ELISA. FIG. 7A shows by C1/6.1 Western blot analysis the relative level of APP holoprotein expression in L cells versus the human APP₆₉₅ overexpressing L cell line (L/APP). In FIG. 7B, lysates prepared from these cells either grown under control conditions or subjected to calpeptin treatment were examined using the C1/6.1-C2/7.1 sandwich ELISA. As expected given the levels of APP detected by Western blot analysis, L/APP cells showed significantly more ELISA signal than did the parental L cells. The addition of calpeptin increased the signal in the L cells, although not to a statistically significant degree. In L/APP cells, however, the addition of calpeptin greatly increased the ELISA signal, as would be expected due to the generation of CTFs in this system. This results confirms that the APP/total CTF ELISA can be used to track changes in the levels of cell-associated APP metabolites generated by a cell.

### Example 5

Differentiating between toxicity and specific effects on βCTF levels. To determine whether the combination of the βCTF ELISA and the APP/total CTF ELISA allowed differentiation between a toxic effect and a specific effect on βCTF levels, the protein synthesis inhibitor cyclohexamide was used to model a toxic compound. Cyclohexamide has many of the characteristics of a compound that would give a false-positive in a standard screen by reducing cell viability and therefore dramatically reducing Aβ generation.

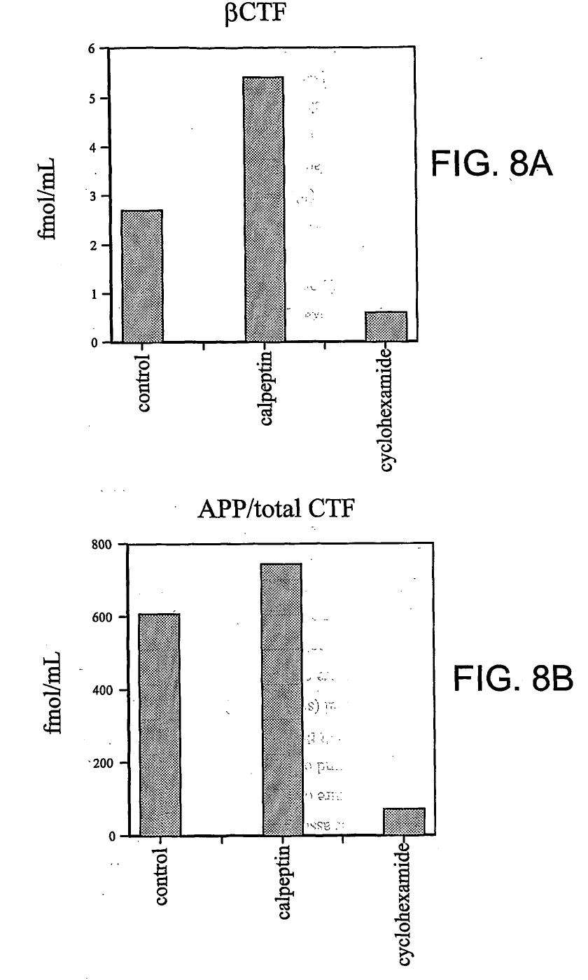

Were a screening protocol to rely upon a reduction in Aβ as the sole outcome measure, cyclohexamide treatment would reduce Aβ production and be seen as a potential therapeutic; however, cyclohexamide is highly toxic. FIGS. 8A and 8B show the results from an experiment in which control L/APP cells were compared with equally dense L/APP cells treated with either 10 μM calpeptin or 75 μg/ml cyclohexamide for 6 hours. FIG. 8A shows βCTF levels and FIG. 8B shows APP holoprotein/CTF levels determined by ELISA. Calpeptin treatment was seen to significantly increase βCTF levels (approximately doubling from 2.7 to 5.4 fmole/ml cell lysate), while showing a smaller relative increase in APP/total CTF levels using the APP/total CTF ELISA. Cyclohexamide treatment reduced βCTF levels (by 78%, from 2.7 fmole/ml to 0.6 fmole/ml) while at the same time dramatically reducing APP/total CTF levels (by 88%, from 608 fmole/ml in control to 74 fmole/ml in cyclohexamide-treated L/APP cells).

These results demonstrate that, in combination, these two ELISAs can be advantageously used to detect a reduction in βCTF levels, while differentiating between compounds that specifically reduce β-cleavage (a desirable outcome) and compounds that reduce βCTF levels via general cell toxicity (e.g., as demonstrated using cyclohexamide).

### Example 6

Detection of a selective inhibitor of βCTF generation using βCTF and APP/total CTF ELISAs according to the present invention. 3-methyl adenine (3MA), an inhibitor of cellular autophagy (P. O. Seglen and P. B. Gordon, 1982, Proc. Natl. Acad. Sci. USA, 79(6):1889-1892; P. E. Schwarze and P. O. Seglen, 1985, Exp. Cell. Res., 157(1):15-28; and P. O. Seglen et al., 1986, Exp. Cell. Res., 162(1):273-277), has been reported to have potential therapeutic value in AD and/or other neuronal, atrophy-associated dementing disorders (patent application U.S. Ser. No. 09/561,582 “Methods for the treatment of neuronal atrophy-associated dementia”; to R. A. Nixon et al., filed Apr. 28, 2000).

This example describes the results of experiments conducted using 3MA as a test compound to demonstrate the present method and its advantages. FIGS. 10A-10D present the results of these experiments which demonstrate a specific reduction by 3MA of βCTF generation and Aβ production in L/APP cells. FIG. 10A shows that 3MA treatment did not reduce cellular APP levels relative to levels of APP in untreated control cells, while cyclohexamide treatment did. FIG. 10B shows that 3MA treatment reduced βCTF levels by 50% relative to control while cyclohexamide treatment reduced βCTF levels somewhat more. These results are consistent with cyclohexamide being a non-specific cellular toxin that reduces total cellular APP levels, thereby also reducing βCTF levels. These results further indicate that the effect of 3MA on βCTF levels is specific, and is not the result of genera cell cytotoxicity. This experiment demonstrates that, according to the present invention, the ELISAs in combination can differentiate between potentially therapeutic compounds (e.g., 3MA) and a toxic compound (e.g., cyclohexamide).

In addition, Aβ levels in the growth media were examined from these test cells. FIG. 10C shows the reduction in Aβ1-40 levels relative to control, which is observed with both 3MA and cyclohexamide treatment. FIG. 10D shows a similar reduction in Aβ1-42 levels relative to control, which is evident with both treatments. Again, the reduction in Aβ generation obtained with cyclohexamide treatment is due to non-specific cytotoxicity, while the reduction in Aβ generation obtained following 3MA treatment is linked to a specific reduction in βCTF generation.

These findings demonstrate the usefulness of the method of the cell-based screening methods of the present invention in which levels of a precursor and levels of a metabolite are both assayed, and, in particular, the use of a βCTF ELISA and an APP/total CTF ELISA involved in AD pathways, to differentiate between generally toxic compounds and compounds with potential therapeutic value using a living cell system.

The contents of all patents, patent applications, published US and PCT applications, articles, books, references, reference manuals, the Sequence Listing and abstracts cited herein are hereby incorporated by reference in their entirety to more fully describe the state of the art to which the invention pertains.

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings.

What is claimed is:

1. A cell-based screening method to identify compounds that reduce or inhibit the generation of one or more cellular metabolites associated with a disease or disorder, without causing non-specific cytotoxicity, comprising:
(a) contacting cells with a test compound;
(b) determining levels of (i) a cellular precursor protein, or (ii) a conformation state of a cellular precursor protein; and
(c) determining levels of a metabolite generated from (i) or (ii) of step (b); wherein a compound that specifically reduces or inhibits the level of the metabolite in cells shows a reduction only in the level of the metabolite relative to that in untreated cells, and a compound that is non-specifically cytotoxic shows a reduction in the levels of (i), and, optionally, associated cleavage products of (i); or of (ii); and of the metabolite.

2. The method according to claim 1, which is a high-throughput screening method.

3. The method according to claim 1 or claim 2, wherein the method is performed in a single well of a microtiter plate using antibodies directed toward specific epitopes of the metabolite and antibodies directed toward specific epitopes of the precursor protein and, optionally, its associated cleavage products.

4. The method according to claim 1, wherein the determination of the levels of the cellular precursor protein is performed by an enzyme linked immunosorbent assay comprising one or more detectable antibodies specific for epitopes of the cellular precursor protein.

5. The method according to claim 1, wherein the determination of the levels of the metabolite generated by the precursor protein is performed by an enzyme linked immunosorbent assay comprising one or more detectable antibodies specific for epitopes of the metabolite.

6. The method according to claim 1, wherein the cellular precursor protein (i) is amyloid precursor protein (APP) and, optionally, the associated cleavage products are α carboxy-terminal fragment (αCTF), β carboxy-terminal fragment (βCTF) and γ carboxy-terminal fragment (γCTF).

7. The method according to claim 1, wherein the metabolite is selected from the group consisting of post-translationally modified cellular precursor protein, protein or peptide oligomers, protein or peptide multimers, protofibrillar macromolecules, fibrillar macromolecules and oligomeric assemblies.

8. The method according to claim 1, wherein the metabolite is a proteolytic product of amyloid precursor protein APP.

9. The method according to claim 8, wherein the metabolite is β carboxy-terminal fragment (βCTF) or α carboxy-terminal fragment (αCTF).

10. The method according to claim 1, wherein the cells contacted with the test compound are selected from primary mammalian cells, mammalian cell lines, neuroblastomas, neurons, isolated brain cells, cells transfected with the gene encoding amyloid precursor protein APP and expressing the encoded APP product, and cells genetically modified to mimic Alzheimer's disease pathobiology.

11. The method according to claim 10, wherein the cells are human cells or human cell lines.

12. The method according to claim 1, wherein the test compound is selected from a synthetic organic compound, a chemical compound, a natural product, a polypeptide, or a peptide.

13. A cell-based screening method to identify compounds that increase or augment the generation of one or more cellular metabolites associated with a decreased likelihood of developing, or with a decreased severity of, a disease or disorder, without causing non-specific cytotoxicity, comprising:
(a) contacting cells with a test compound;
(b) determining levels of (i) a cellular precursor protein, or (ii) a conformation state of a cellular precursor protein; and
(c) determining levels of a metabolite generated from (i) or (ii) of step (b); wherein a compound that specifically increases or augments the level of the metabolite in cells shows an increase in the levels of the metabolite relative to untreated cells, while not significantly changing the levels of (i) or (ii).

14. The method according to claim 13, which is a high-throughput screening method.

15. The method according to claim 13 or claim 14, wherein the method is performed in a single well of a microtiter plate using antibodies directed toward specific epitopes of the metabolite and antibodies directed toward specific epitopes of the precursor protein and, optionally, its associated cleavage products.

16. The method according to claim 13, wherein the determination of the levels of the cellular precursor protein is performed by an enzyme linked immunosorbent assay comprising one or more detectable antibodies specific for epitopes of the cellular precursor protein.

17. The method according to claim 13, wherein the determination of the levels of the metabolite generated by the precursor protein is performed by an enzyme linked immunosorbent assay comprising one or more detectable antibodies specific for epitopes of the metabolite.

18. The method according to claim 15, wherein the cellular precursor protein is amyloid precursor protein (APP), or other proteolytic fragment of APP, and, optionally, the associated cleavage products are α carboxy-terminal fragment (αCTF), β carboxy-terminal fragment (βCTF) and γ carboxy-terminal fragment (γCTF).

19. The method according to claim 13, wherein the metabolite is selected from the group consisting of post-translationally modified cellular precursor protein, protein or peptide oligomers, protein or peptide multimers, protofibrillar macromolecules, fibrillar macromolecules and oligomeric assemblies.

20. The method according to claim 13, wherein the metabolite is a proteolytic product of amyloid precursor protein APP.

21. The method according to claim 20, wherein the metabolite is secretory APP or α carboxy-terminal fragment (αCTF) of APP.

22. The method according to claim 13, wherein the cells contacted with the test compound are selected from primary mammalian cells, cultured mammalian cell lines, neuroblastomas, neurons, isolated brain cells, cells transfected with the gene encoding amyloid precursor protein APP and expressing the encoded APP product, and cells genetically modified to mimic Alzheimer's disease pathobiology.

23. The method according to claim 22, wherein the cells are human cells or human cell lines.

24. The method according to claim 13, wherein the test compound is selected from a synthetic organic compound, a chemical compound, a natural product, a polypeptide, or a peptide.

25. A cell-based screening method for distinguishing between a compound that specifically reduces activity of cellular proteolytic pathways involved in metabolic events associated with Alzheimer's disease, and a compound that is non-specifically toxic to cells, comprising:
(a) treating cells with a test compound;
(b) detecting levels of one or more amyloid precursor protein (APP) metabolites;
(c) detecting levels of amyloid precursor protein (APP);
(d) comparing the effect of the compound on the levels of the one or more amyloid precursor protein (APP) metabolites and the effect of the compound on the levels of amyloid precursor protein (APP);
wherein a specific reduction in the levels of the one or more amyloid precursor protein (APP) metabolites, with no significant reduction in the levels of the amyloid precursor protein (APP), indicates that the compound specifically inhibits cellular proteolytic pathways and is not non-specifically cytotoxic.

26. The method according to claim 25, which is a high-throughput screening method.

27. The method according to claim 25, wherein, in step (b), the one or more amyloid precursor protein (APP) metabolites is cell-associated β carboxy-terminal fragment (βCTF) resulting from proteolytic processing of amyloid precursor protein APP.

28. The method according to claim 25, further wherein, in step (c), proteolytic fragments comprising α carboxy-terminal fragment (αCTF), β carboxy-terminal fragment (βCTF) and γ carboxy-terminal fragment (γCTF) of the amyloid precursor protein (APP) are detected.

29. The method according to claim 25 or 26, wherein the method is performed in a single well of a microtiter plate using antibodies directed toward specific epitopes of the one or more amyloid precursor protein (APP) metabolites and antibodies directed toward specific epitopes of amyloid precursor protein (APP).

30. The method according to claim 25, wherein the determination of the levels of the one or more amyloid precursor protein (APP) metabolites is performed by an enzyme linked immunosorbent assay comprising one or more detectable antibodies specific for epitopes on the one or more APP metabolites.

31. The method according to claim 25, wherein the determination of the levels of the amyloid precursor protein (APP) is performed by an enzyme linked immunosorbent assay comprising one or more detectable antibodies specific for epitopes on the amyloid precursor protein (APP).

32. The method according to claim 25, wherein the cells contacted with the test compound are selected from primary mammalian cells, cultured mammalian cell lines, neuroblastomas, neurons, isolated brain cells, cells transfected with the gene encoding amyloid precursor protein APP and expressing the encoded APP product, and cells genetically modified to mimic Alzheimer's disease pathobiology.

33. The method according to claim 32, wherein the cells are human cells or human cell lines.

34. The method according to claim 25, wherein the test compound is selected from a synthetic organic compound, a chemical compound, a natural product, a polypeptide, or a peptide.

35. A cell-based screening method for distinguishing between a compound that specifically reduces activity of cellular proteolytic pathways involved in metabolic events associated with Alzheimer's disease, and a compound that is non-specifically toxic to cells, comprising:
(a) treating cells with a test compound;
(b) detecting levels of cell-associated β carboxy-terminal fragment (βCTF) resulting from proteolytic processing of amyloid precursor protein APP;
(c) detecting levels of amyloid precursor protein (APP) and its associated cleavage fragments comprising α carboxy-terminal fragment (αCTF), β carboxy-terminal fragment (βCTF) and γ carboxy-terminal fragment (γCTF);
(d) comparing the effect of the compound on the levels of βCTF generated from the cleavage of APP and the effect of the compound on the levels of amyloid precursor protein (APP) and its associated cleavage, fragments; wherein a specific reduction in the levels of βCTF, with no significant reduction in the levels of amyloid precursor protein (APP) and its associated cleavage fragments relative to control, indicates that the compound specifically inhibits cellular proteolytic pathways and is not non-specifically cytotoxic.

36. The method according to claim 35, which is a high-throughput screening method.

37. The method according to claim 35 or claim 36, wherein the method is performed in a single well of a microtiter plate using antibodies directed toward specific epitopes of the β carboxy-terminal fragment (βCTF) and antibodies directed toward specific epitopes of amyloid precursor protein (APP) and its associated cleavage fragments.

38. The method according to claim 35, wherein the determination of the levels of the cell-associated β carboxy-terminal fragment (βCTF) resulting from the proteolytic processing of amyloid precursor protein APP is performed by an enzyme linked immunosorbent assay comprising one or more detectable antibodies specific for epitopes on the cell-associated β carboxy-terminal fragment (βCTF).

39. The method according to claim 35, wherein the determination of the levels of the amyloid precursor protein (APP) and its associated cleavage fragments is performed by an enzyme linked immunosorbent assay comprising one or more detectable antibodies specific for epitopes on the amyloid precursor protein (APP) and its associated cleavage fragments.

40. The method according to claim 35, wherein the cells treated with the test compound are selected from primary mammalian cells, cultured mammalian cell lines, neuroblastomas, neurons, isolated brain cells, cells transfected with the gene encoding amyloid precursor protein APP and expressing the encoded APP product, and cells genetically modified to mimic Alzheimer's disease pathobiology.

41. The method according to claim 40, wherein the cells are human cells or human cell lines.

42. The method according to claim 35, wherein the test compound is selected from a synthetic organic compound, a chemical compound, a natural product, a polypeptide, or a peptide.

43. A cell-based screening method to identify compounds that reduce or inhibit the activity of cellular metabolites involved in an Alzheimer's disease metabolic pathway, comprising:
(a) treating with a test compound cells plated onto a substrate coated with one or more capture antibodies directed against amyloid precursor protein (APP) and APP cell associated metabolites;
(b) disrupting the cells, wherein extracted amyloid precursor protein (APP) and APP cell associated metabolites bind to the one or more capture antibodies;
(c) detecting binding of protein to capture antibody with detection antibodies, wherein the detection antibodies comprise (i) labeled antibodies that specifically recognize one or more APP metabolites and (ii) differently labeled antibodies that specifically recognize amyloid precursor protein (APP) and, optionally, carboxy-terminal, proteolytic products thereof; and
(d) determining the levels of (i) the one or more APP metabolites, and (ii) the amyloid precursor protein (APP) and, optionally, the carboxy-terminal proteolytic products thereof; wherein a decrease in the levels of the one or more APP metabolites, without a significant decrease in the level of the APP protein, and optionally, the carboxy-terminal proteolytic products thereof, relative to untreated cells, indicates that the compound specifically inhibits cellular proteolytic pathways and is not non-specifically cytotoxic.

44. The method according to claim 43, which is a high-throughput screening method.

45. The method according to claim 43, wherein the capture antibody is directed against the carboxy-terminus of the amyloid precursor protein (APP).

46. The method according to claim 43, wherein the one or more APP metabolites is selected from βCTF, αCTF and γCTF.

47. The method according to claim 43, wherein the one or more APP metabolites is βCTF.

48. The method according to claim 43, wherein the cells treated with the test compound are selected from primary mammalian cells, cultured mammalian cell lines, neuroblastomas, neurons, isolated brain cells, cells transfected with the gene encoding amyloid precursor protein APP and expressing the encoded APP product, and cells genetically modified to mimic Alzheimer's disease pathobiology.

49. The method according to claim 48, wherein the cells are human cells or human cell lines.

50. The method according to claim 43, wherein the test compound is selected from a synthetic organic compound, a chemical compound, a natural product, a polypeptide, or a peptide.

51. The method according to claim 43, wherein the detection antibodies are labeled with different fluorophores.

52. The method according to any one of claims 1, 13, 25, 35 and 43, wherein the precursor and the metabolite are detected with labeled antibodies.

53. The method according to claim 52, wherein the antibodies are labeled with a fluorophore, an enzyme, a chemiluminescent molecule, or a radioisotope.

54. A compound that specifically reduces the generation of a metabolite involved in a metabolic pathway associated with disease progression, detected according to the method of claim 1 or claim 13.

55. A compound that specifically increases the generation of a metabolite involved in a metabolic pathway associated with disease progression, detected according to the method of claim 1 or claim 13.

56. A compound that specifically reduces the generation of a metabolite in a proteolytic metabolic pathway associated with Alzheimer's disease, as detected by the method according to claim 1, claim 13, claim 25, claim 35, or claim 43.

57. The compound according to claim 56, said compound being 3-methyladenine or functionally related derivatives or analogs thereof.

58. A compound that specifically increases the generation of a metabolite in a proteolytic metabolic pathway associated with Alzheimer's disease, as detected by the method according to claim 1, claim 13, claim 25, claim 35, or claim 43.

59. The compound according to claim 58, said compound being αCTF.

60. A kit for detecting compounds which specifically reduce or increase a cellular metabolite in a pathway associated with a disease or disorder, and which are not generally toxic to cells, comprising containers comprising one or more detectable reagents to determine levels of (i) a cellular metabolic precursor protein or (ii) a conformation state of a cellular precursor protein; and (iii) one or more metabolites of the precursor protein of (i) or (ii); and instructions for use.

61. The kit according to claim 60, wherein the detectable reagents comprise one or more labeled or unlabeled antibodies specific for the metabolic precursor protein and labeled or unlabeled antibodies specific for the one or more metabolites; and further comprising one or more labeled secondary antibodies for differential detection of the unlabeled specific antibodies.
